SEQUENCE LISTING (48 sequences)

SEQ ID NO: 1 - DNA, 200 bp, Mus musculus
gatctccagatggctaaacttttaaatcatgaatgaagtagatattaccaaattgctttttcagcatccatttagataatcatgttttttgcctttaatctgttaatgtagtgaattacagaaatacatttcctaaatcattacatcccccaaatcgttaatctgctaaagtacatctctggctcaaacaagactggttg

SEQ ID NO: 2 - DNA, 445 bp, Mus musculus
gcatcttgttcctttgggcgcatggctgatgaccacattcatccttttaatctttcactcactaaaggtcaggcttagatgagtgttcaaaatgcttgttggtacagaaaatgattggaggcctcaccctaatccctctttcaaaatgtactatccaattccattctggggataaaagagtttcagaggcccacattggctccccctcccatctcactccagccataaatgggaggtaggaagagctctaatccctccactgtctaagtagaattttccatgagctgtcacagatctctcacagggactgaggaagaagctaagagaaacaggaggagttggcaaaggattgagacaatcaggaagctgggttgcctgctgcaaggaagtaggaaggaggggccatcttgacatcagagcttggtgaacactacctgctccaatg

SEQ ID NO: 3 - DNA, 164 bp, Mus musculus
gagaagagcactggctggggctgcttgcccgctaatcccagctgccattaaaatattaaagataaatctaatcgtctctttatccaaaataagcgacttttgtgtggggagaaaacgtctaaccccttaggaggagaattagttctaatgcatcaaatggaatt

SEQ ID NO: 4 - DNA, 13 bp, Mus musculus
ccctaatccctct

SEQ ID NO: 5 - DNA, 16 bp, Mus musculus
tctttcaaaatgtact

SEQ ID NO: 6 - DNA, 13 bp, Mus musculus
ctctaatccctcc

SEQ ID NO: 7 - DNA, 16 bp, Mus musculus
agaattttccatgagc

SEQ ID NO: 8 - DNA, 13 bp, Mus musculus
ccgctaatcccag

SEQ ID NO: 9 - DNA, 10 bp, Mus musculus
ttaaaatatt

SEQ ID NO: 10 - DNA, 8 bp, Mus musculus
ctaatcgt

SEQ ID NO: 11 - DNA, 16 bp, Mus musculus
ttatccaaaataagcg

SEQ ID NO: 12 - DNA, 19 bp, Artificial Sequence
  Amplification Primer
gatctccagatggctaaac

SEQ ID NO: 13 - DNA, 20 bp, Artificial Sequence
  Amplification Primer
ggcggacgaagctgccaccc

SEQ ID NO: 14 - DNA, 20 bp, Artificial Sequence
  Amplification Primer
ggctcaaacaagactggttg

SEQ ID NO: 15 - DNA, 20 bp, Artificial Sequence
  Amplification Primer
ggcggacgaagctgccaccc

SEQ ID NO: 16 - DNA, 19 bp, Artificial Sequence
  Amplification Primer
ccaagcttattggtgttgc

SEQ ID NO: 17 - DNA, 20 bp, Artificial Sequence
  Amplification Primer
ggcggacgaagctgccaccc

SEQ ID NO: 18 - DNA, 19 bp, Artificial Sequence
  Amplification Primer
gatctccagatggctaaac

SEQ ID NO: 19 - DNA, 20 bp, Artificial Sequence
  Amplification Primer
caaccagtcttgtttgagcc

SEQ ID NO: 20 - DNA, 20 bp, Artificial Sequence
  Amplification Primer
gcatcttgttcctttgggcg

SEQ ID NO: 21 - DNA, 17 bp, Artificial Sequence
  Amplification Primer
cattggagcaggtagtg

SEQ ID NO: 22 - DNA, 20 bp, Artificial Sequence
  Amplification Primer
gagaagagcactggctgggg

SEQ ID NO: 23 - DNA, 35 bp, Artificial Sequence
  Amplification Primer
aattccatttgatgcattagaactaattctcctcc

SEQ ID NO: 24 - DNA, 23 bp, Artificial Sequence
  Amplification Primer
catggcgaccgcagcgtctaacc

SEQ ID NO: 25 - DNA, 21 bp, Artificial Sequence
  Amplification Primer
tcactggacgggcgtctgcac

SEQ ID NO: 26 - DNA, 20 bp, Artificial Sequence
  Amplification Primer
gtgtgaggggacctatttcc

SEQ ID NO: 27 - DNA, 20 bp, Artificial Sequence
  Amplification Primer
caagatctgaaacttccagg

SEQ ID NO: 28 - DNA, 32 bp, Artificial Sequence
  Myc tag
gaacaaaaacttatttctgaagaagatctgtg

SEQ ID NO: 29 - DNA, 20 bp, Artificial Sequence
  Amplification Primer
ctggaacgtggaggaagctg

SEQ ID NO: 30 - DNA, 20 bp, Artificial Sequence
  Amplification Primer
caaaacctggaatttccatg

SEQ ID NO: 31 - DNA, 33 bp, Artificial Sequence
  Amplification Primer
ccatgggcacgccgacctcagacgacctggagc

SEQ ID NO: 32 - DNA, 29 bp, Artificial Sequence
  Amplification Primer
accggtccgggaggggtcatccttttctc

SEQ ID NO: 33 - DNA, 33 bp, Mus musculus
ctagctaaacttttaaatcatgaatgaagtaga

SEQ ID NO: 34 - DNA, 24 bp, Mus musculus
ctagctttaatctgttaatgtagt

SEQ ID NO: 35 - DNA, 25 bp, Mus musculus
ctagcaaatcgttaatctgctaaag

SEQ ID NO: 36 - DNA, 25 bp, Mus musculus
ctagcctcaccctaatccctctttc

SEQ ID NO: 37 - DNA, 26 bp, Mus musculus
ctagccctctttcaaaatgtactatc

SEQ ID NO: 38 - DNA, 24 bp, Mus musculus
ctagagagctctaatccctccact

SEQ ID NO: 39 - DNA, 28 bp, Mus musculus
ctagtaagtagaattttccatgagctgt

SEQ ID NO: 40 - DNA, 24 bp, Mus musculus
ctagttgcccgctaatcccagctg

SEQ ID NO: 41 - DNA, 24 bp, Mus musculus
ctagctgccattaaaatattaaag

SEQ ID NO: 42 - DNA, 24 bp, Mus musculus
ctagagataaatctaatcgtctct

SEQ ID NO: 43 - DNA, 28 bp, Mus musculus
ctagtctctttatccaaaataagcgact

SEQ ID NO: 44 - DNA, 24 bp, Mus musculus
acttttaaatcatgaatgaagtag

SEQ ID NO: 45 - DNA, 26 bp, Artificial Sequence
  Amplification Primer
gactagtgatctccagatggctaaac

SEQ ID NO: 46 - DNA, 29 bp, Artificial Sequence
  Amplification Primer
ccgctcgagcaaccagtcttgtttgagcc

SEQ ID NO: 47 - DNA, 10 bp, Mus musculus
ctgttaatgt

SEQ ID NO: 48 - DNA, 13 bp, Mus musculus
cgttaatctgcta

(19) United States
(12) Patent Application Publication
(10) Pub. No.: US 2009/0281163 A1
(43) Pub. Date: Nov. 12, 2009

(54) REGULATORY ELEMENTS THAT MEDIATE RETINAL CELL-SPECIFIC GENE EXPRESSION

(75) Inventors: Constance L. Cepko, Newton, MA (US); Douglas S. Kim, North Bethesda, MD (US); Takahiko Matsuda, Brookline, MA (US)

Correspondence Address: BANNER & WITCOFF, LTD., 28 STATE STREET, 28th FLOOR, BOSTON, MA, 02109-9601, US

(73) Assignee: President and Fellows of Harvard College, Cambridge, MA (US)

(21) Appl. No.: 12/117,812

(22) Filed: May 9, 2008

(51) Int. Cl. A61K 31/711; C07H 21/04; C12N 5/00; C12N 15/63

(52) U.S. Cl. 514/44 A; 536/23.5; 435/325; 435/320.1

(57) ABSTRACT

Isolated nucleic acid molecules including regulatory elements that direct retinal-cell specific expression are provided. Methods for treating or preventing retinal disorders in a subject are also provided.

### STATEMENT OF GOVERNMENT INTERESTS

This invention was made with government support under National Institutes of Health grant numbers R01 EY009676, F32 EY15360 and T32 EY007145. The Government has certain rights in the invention.

### FIELD

The present invention relates to regulatory elements that mediate gene expression in specific retinal cell types.

### BACKGROUND

Retinal function is carried out by diverse cell types that exhibit distinct morphology, connectivity and physiology. The diversity of retinal cell types is also evident in the considerable gene expression heterogeneity observed in developing and mature retinal cells (Blackshaw et al., 2004; Gray et al., 2004; Trimarchi et al., 2007). The mechanisms underlying molecular diversity of retinal cells could be further revealed by examining transcriptional programs that orchestrate gene expression in specific cell types. Bipolar cells are the first relay interneurons in the visual system. They connect rod and cone photoreceptor cells to amacrine and ganglion cells and are critical in processing and routing of visual signals. Bipolar cells express unique combinations of molecules important for form and function, but the transcriptional mechanisms regulating bipolar cell gene expression remain largely unknown (Kim et al., 2007; Bramblett et al., 2004; Ghosh et al., 2004; Pignatelli and Strettoi, 2004; Haverkamp et al., 2003a; Haverkamp et al., 2003b; Huang et al., 2003; Chow et al., 2001; Ohtoshi et al., 2001; Baas et al., 2000; Haeseleer et al., 2000; Koulen et al., 1998; Fletcher et al., 1998; Vardi, 1998; Vardi and Morigiwa, 1997; Takebayashi et al., 1997; Burmeister et al., 1996; Euler and Wässle, 1995; Berrebi et al., 1991; Greferath et al., 1990).

Previous studies have examined retinal phenotypes resulting from mutation of transcription factor genes expressed within bipolar cells and/or their progenitor cells in mice. Several transcription factors expressed in progenitor cells are required individually or in combination for genesis of bipolar cells in general, including Chx10, Mash1, Math3, and Ngn2 (Burmeister et al., 1996; Green et al., 2003; Livne-Bar et al., 2006; Tomita et al., 2000; Akagi et al., 2004). Other transcription factors that initiate expression in exiting or post-mitotic cells in the developing retina have been shown to play important roles in driving differentiation and/or survival of various types of bipolar cells, including Otx2, Crx, Vsx1, Isl1, Irx5, Bhlhb4, and Bhlhb5 (Chow et al., 2004; Ohtoshi et al., 2004; Clark et al., 2007; Elshatory et al., 2007; Cheng et al., 2005; Feng et al., 2006; Bramblett et al., 2004). Despite the elucidation of the roles of many individual transcription factors in bipolar cell development, relationships among transcription factors and their targets have not been defined.

### SUMMARY

The present invention is based in part on the surprising discovery of relatively small (e.g., 164, 200 or 445 base pair (bp) regions as compared with 9.5 kilobase (kb) pair region described (Nakajima et al. (1993) J. Biol. Chem. 268:11868)) regulatory regions that mediate bipolar retinal cell-specific gene expression. The novel regulatory regions described herein are particularly useful, for example, for driving cell- and/or tissue-specific expression of nucleic acid and/or amino acid sequences, e.g., gene therapy in the eye and/or to drive expression of transynaptic neuronal tracing molecules, toxins and/or activity-altering ion channels in various subtypes of bipolar cells. Cis-regulatory elements (CREs) for the bipolar cell-enriched and bipolar cell-specific genes metabotropic glutamate receptor 6 (Grm6), calcium-binding protein 5 (Cabp5), and C. elegans ceh-10 homeodomain containing homolog (Chx10), were defined by in vivo retinal transfection of CRE-reporter DNA constructs.

The diversity of cell types found within the vertebrate central nervous system arises in part from action of complex transcriptional programs. In the retina, the programs driving diversification of various cell types have not been completely elucidated. To investigate gene regulatory networks that underlie formation and function of one retinal circuit component, the bipolar cell, transcriptional regulation of three bipolar cell-enriched genes was analyzed. Using in vivo retinal DNA transfection and reporter gene constructs, a 200-bp Grm6 enhancer sequence (SEQ ID NO: 1), a 445-bp Cabp5 promoter sequence (SEQ ID NO:2), and a 164-bp Chx10 enhancer sequence (SEQ ID NO:3) were defined, each driving reporter expression specifically in distinct but overlapping bipolar cell subtypes. Bioinformatic analysis of sequences revealed the presence of potential paired-type and POU homeodomain-containing transcription factor binding sites (TFBSs), which were shown to be critical for reporter expression through deletion studies. The paired-type homeodomain transcription factors, Crx and Otx2, and POU homeodomain factor, Brn2, are expressed in bipolar cells and interacted with the predicted binding sequences as assessed by electrophoretic mobility shift assay (EMSA). Grm6, Cabp5, and Chx10 reporter activity and endogenous gene expression were both reduced in Otx2 loss-of-function retinas. Expression of several other bipolar cell molecular markers was also dependent on paired-type homeodomain-containing transcription factors, as assessed by RNA in situ hybridization in mutant retinas. Cabp5 and Chx10 reporter expression was reduced in dominant-negative Brn2-transfected retinas. The paired-type and POU homeodomain-containing transcription factors, Otx2 and Brn2, together appear to play a common role in regulating genes involved in both bipolar cell fate determination and differentiation.

Accordingly, in certain exemplary embodiments, an isolated nucleic acid molecule comprising a nucleotide sequence which is at least 70%, 80%, 90%, 92% or 95% identical to the nucleotide sequence of SEQ ID NO:1, 2 or 3 is provided. In certain exemplary embodiments, an isolated nucleic acid molecule comprising a nucleotide sequence set forth as SEQ ID NO:1, 2 or 3 is provided. In certain exemplary embodiments, a nucleic acid molecule comprising a fragment of at least 15 nucleotides of the nucleotide sequence of SEQ ID NO: 1, 2 or 3 is provided.

In certain aspects of these embodiments, the isolated nucleic acid molecule is capable of directing bipolar cell-specific expression, e.g., in the retina. In other aspects of these embodiments, the isolated nucleic acid molecule has one or more POU homeodomain binding sites and/or one or more paired-type homeodomain binding sites. In certain aspects of these embodiments, the nucleic acid molecule includes a basal promoter sequence. In certain aspects of these embodiments, the isolated nucleic acid molecule is capable of directing bipolar cell-specific expression of a nucleic acid sequence of interest, e.g., one or more of an endogenous retinal mRNA sequence, a heterologous mRNA sequence, an antisense RNA sequence, an RNAi sequence and/or an siRNA sequence. In certain aspects of these embodiments, a host cell contains the isolated nucleic acid molecule. In other aspects of these embodiments, the isolated nucleic acid molecule is provided in a gene therapy vector.

In certain exemplary embodiments, a method for treating a subject in need thereof including administering to the subject an isolated nucleic acid molecule described above, expressing a nucleic acid sequence of interest in a retinal cell, and treating or preventing a retinal disorder is provided. In certain aspects, the retinal disorder is selected from the group consisting of blindness, atrophic macular degeneration, retinitis pigmentosa, iatrogenic retinopathy, retinal tears and holes, diabetic retinopathy, sickle cell retinopathy, retinal vein occlusion, retinal artery occlusion and aberrant cell proliferation, e.g., retinal cancer. In certain aspects, the nucleic acid molecule is administered using a gene therapy vector. In other aspects, the gene therapy vector is a viral vector. In certain aspects, the nucleic acid molecule includes a basal promoter sequence. In still other aspects, the nucleic acid molecule is administered by in vivo electroporation.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present invention will be more fully understood from the following detailed description of illustrative embodiments taken in conjunction with the accompanying drawings in which:

FIGS. 1A-1P depict Grm6 CRE isolation. Representative sections from in vivo neonatal rat retinal transfections with Grm6-LacZ and UB-GFP or CAG-GFP constructs are shown. Retinas were harvested at P15-P22. (A), (C), (E), (G), (I), (K), (L), (M), (O), X-gal (dark blue) and DAPI (light blue) staining. (B), (D), (F), (H), (J), (L), (N), (P), GFP fluorescence (green) and DAPI staining (blue) in same section. (A) 10-kb 5═ flanking mouse genomic sequence Grm6-LacZ transfection (Ueda et al., 1997). (B) UB-GFP co-transfection. (C) Bg/II deletion construct-LacZ transfection. (D) CAG-GFP co-transfection. (E) MscI deletion construct-LacZ transfection. (F) CAG-GFP co-transfection. (G) 1-kb critical region-3′ 0.5-kb sequence-LacZ transfection. (H) CAG-GFP co-transfection. (1) 1-kb critical region without conserved 5′ sequence-3′ 0.5-kb sequence-LacZ transfection. (J) CAG-GFP co-transfection. (K) 3′ 0.5-kb sequence-LacZ transfection. (L) CAG-GFP co-transfection. (M) 200-bp Grm6-SV40 promoter-LacZ transfection. (N) UB-GFP co-transfection. (O) SV40 promoter-LacZ transfection. (P) UB-GFP co-transfection. Grm6 partial mouse genomic structure is shown in light blue. 10-kb 5′ flanking genomic sequence is shown in purple. Conservation of syntenic regions of genomes of several species is plotted in dark blue. Pairwise comparison of mouse sequence and syntenic regions of other species is plotted below. Numbers in black are sequence positions relative to the first nucleotide of BC021919 (GenBank, NIH). 1-kb critical region (green). 200-bp CRE (red). S: SphI restriction enzyme site. B: BglII. M: MscI. N: NaeI. ONL: outer nuclear layer. INL: inner nuclear layer. GCL, ganglion cell layer. Scale bar: 100 μm. Numbers in gray represent nucleotide positions on mouse chromosome 11, according to coordinates based on the February 2006 (mm8) mouse genome assembly from the UCSC Genome Browser Project (Santa Cruz, Calif.; Kent et al., 2002). When viewing this figure, place sheet 2/26 to the right of sheet 1/26, and place sheet 4/26 to the right of sheet 3/26.

FIGS. 2A-2F depict Cabp5 CRE isolation. Representative sections from in vivo neonatal mouse retinal transfections with Cabp5-tdTomato and Cabp5-GFP constructs are shown. Retinas were harvested at P14. (A) 4.7-kb 5′ flanking mouse genomic sequence-tdTomato transfection (Matsuda and Cepko, 2004). (B) 4.7-kb 5′ flanking mouse genomic sequence-GFP transfection. (C) Merged images, tdTomato fluorescence (red), GFP fluorescence (green), DAPI staining (blue). (D) 4.7-kb 5′ flanking mouse genomic sequence-tdTomato transfection. (E) 445-bp Cabp5-GFP transfection. (F) Merged images. Cabp5 partial mouse genomic structure is shown in dark blue. 4.7-kb 5′ flanking genomic sequence is shown as black rectangle. Conservation of syntenic regions of genomes of several species is plotted in dark blue. Pairwise comparison of mouse sequence and syntenic regions of other species is plotted below. Numbers in black are sequence positions relative to the first nucleotide of NM_{—}013877. Scale bar: 100 μm. Numbers in gray represent nucleotide positions on mouse chromosome 11, according to coordinates based on the February 2006 (mm8) mouse genome assembly from the UCSC Genome Browser Project (Santa Cruz, Calif.; Kent et al., 2002). When viewing this figure, place sheet 6/26 to the right of sheet 5/26.

FIGS. 3A-3E depict Chx10 CRE screening and isolation. (A) schematically depicts an unbiased CRE screening scheme. Reporter vector contains a cloning site inserted upstream of an SV40 basal promoter and GFP. A mouse Chx10 BAC was digested with EcoRI and fragments were used to construct a CRE library. (B) Representative section from in vitro neonatal mouse transfection of a CRE clone containing five genomic fragments. Retina was harvested after 9 d of culture. GFP fluorescence (green) and DAPI staining (blue). (C) Representative sections from in vivo neonatal rat transfection of positive CRE clone. Retinas were harvested at P14. (D) 2.5-kb Chx10-SV40 promoter-tdTomato transfection. Retinas were harvested at P14. tdTomato fluorescence (red). (E) 164-bp Chx10-SV40 promoter-GFP-IRES-AP transfection. Retinas were harvested at P14. BCIP/NBT staining (dark purple). Chx10 partial mouse genomic structure is shown in dark blue. 2.5-kb genomic sequence is shown in purple. Conservation of syntenic regions of genomes of several species is plotted in dark blue. Pairwise comparison of mouse sequence and syntenic regions of other species is plotted below. Numbers in black are sequence positions relative to the first nucleotide of NM_{—}007701. Scale bars: 100 μm. Numbers in gray represent nucleotide positions on mouse chromosome 11, according to coordinates based on the February 2006 (mm8) mouse genome assembly from the UCSC Genome Browser Project (Santa Cruz, Calif.; Kent et al., 2002). When viewing this figure, place sheet 9/26 to the right of sheet 8/26.

FIGS. 4A-4S depict Grm6 CRE deletion analysis. Representative sections from in vivo neonatal mouse retinal transfections with the 200-bp Grm6-SV40 promoter-tdTomato construct and Grm6-SV40 promoter-GFP deletion constructs are shown. Retinas were harvested at P14-P21. (A), (D), (G), (J), (M), (P), 200-bp Grm6-SV40 promoter-tdTomato transfection. (C), (F), (I), (L), (O), (R), Merged images, tdTomato fluorescence (red), GFP fluorescence (green), DAPI staining (blue). (B) 200-bp Grm6-SV40 promoter-GFP transfection. (E) Pax6 site deletion. (H) Pou3f2 site deletion. (K) Crx site deletion. (N) Pou3f2 and Crx site deletion. (Q) SV40 promoter-GFP transfection. (S) Sequence alignment of mouse 200-bp Grm6 CRE and syntenic sequence from rat, human, and dog genomes. Asterisks denote conserved nucleotides. Red box: Pax site. Green box: Pou3f2 site. Blue box: Crx site. Scale bar: 100 μm.

FIGS. 5A-5S depict Cabp5 CRE deletion analysis. Representative sections from in vivo neonatal mouse retinal transfections with the 445-bp Cabp5-tdTomato construct and Cabp5-GFP deletion constructs are shown. Retinas were harvested at P14. (A), (D), (G), (J), (M), (P), 445-bp Cabp5-tdTomato transfection. (C), (F), (I), (L), (O), (R), Merged images, tdTomato fluorescence (red), GFP fluorescence (green), DAPI staining (blue). (B) 445-bp Cabp5-GFP transfection. (E) Crx site deletion. (H) 5′ Bm2 site deletion. (K) Pitx2 site deletion. (N) 3′ Bm2 site deletion. (Q) 5′ Brn2 and 3′ Brn2 site deletion. (S) Sequence alignment of mouse 445-bp Cabp5 CRE and syntenic sequence from rat, human, and dog genomes. Asterisks denote conserved nucleotides. Red box: Crx site. Green box: 5′ Bm2 site. Blue box: Pitx2 site. Purple box: 3′ Brn2 site. Scale bar: 100 μm.

FIGS. 6A-6M depict Chx10 CRE deletion analysis. Representative sections from in vivo neonatal mouse retinal transfections with the 164-bp Chx10-SV40 promoter-GFP-IRES-AP construct, Chx10-SV40 promoter-GFP deletion constructs, and UB-tdTomato or UB-GFP constructs are shown. Retinas were harvested at P14-P21. (A), (C), (E), (G), (I), (K), BCIP/NBT (dark purple) and DAPI (light blue) staining. (B), (D), (F), (H), (J), (L), tdTomato fluorescence (red), GFP fluorescence (green), DAPI staining (blue). (A) 164-bp Chx10-SV40 promoter-GFP-IRES-AP transfection. (C) Crx site deletion. (E) Pou3f2 site deletion. (G) Otx site deletion. (I) Brn2 site deletion. (K) SV40 promoter-GFP-IRES-AP transfection. (M) Sequence alignment of mouse 164-bp Chx10 CRE and syntenic sequence from rat, human, dog, opossum, and chicken genomes. Asterisks denote conserved nucleotides. Red box: Crx site. Green box: Pou3f2 site. Blue box: Otx site. Purple box: Brn2 site. Scale bar: 100 μm.

FIGS. 7A-7I depict electrophoretic mobility shift assay (EMSA) analyses. Autoradiograms of EMSAs using nuclear extract from 293T cells transfected with CAG-GFP, CAG-Brn2, CAG-CrxMyc, or CAG-Otx2Myc (A-C) and nuclear extract from adult mouse retinas (F-H) are shown. (A), (F), EMSA results using oligonucleotide probes overlapping the Grm6 Pax6, Pou3f2, and Crx sites. Negative control reactions without nuclear extract (−). Experimental reactions with nuclear extract (+NE). (B), (G), EMSA results using oligonucleotide probes overlapping the Cabp5 Crx, 5′ Brn2, Pitx2, and 3′ Brn2 sites. (C), (H), EMSA results using oligonucleotide probes overlapping the Chx10 Crx, Pou3f2, Otx, and Brn2 sites. (D) Western blot analysis of nuclear extracts using an anti-Brn2 or anti-Myc antibody. Numbers denote molecular weight in kD. (E) Sequence alignment of oligonucleotides overlapping the Grm6, Cabp5, and Chx10 sites. Underlined sequences are TAAT sequences or closest matches. Summary of interactions with transcription factors from two families is listed on right. POU: POU homeodomain-containing transcription factor interaction. PHD: paired-type homeodomain-containing transcription factor interaction. (I) Summary of interactions.

FIGS. 8A-8R depict an Otx2 conditional loss-of-function analysis. Representative sections from in vivo neonatal retinal transfections of Otx2^{flox/flox} mice with UB-GFP, reporter constructs, and CAG-Cre are shown. Retinas were harvested at P14-P15. (A)-(C), (G)-(I), (M)-(O), Control transfection without CAG-Cre. (D)-(F), (J)-(L), (P)-(R), Otx2 conditional loss-of-function resulting from CAG-Cre transfection. (B), (E), (H), (K), (M), (Q), UB-GFP transfection. (C), (F), (I), (L), (O), (R), Merged images, tdTomato fluorescence (red), GFP fluorescence (green), DAPI staining (blue). (A), (D), 200-bp Grm6-SV40 promoter-tdTomato transfection. G, J, 445-bp Cabp5-tdTomato transfection. (M), (P), 164-bp Chx10-SV40 promoter-tdTomato transfection. Circles: UB-GFP-transfected bipolar cells. Scale bar: 100 μm.

FIGS. 9A-9R depict dominant negative Brn2 effects. Representative sections from in vivo neonatal retinal transfections of wild-type mice with UB-GFP, reporter constructs, CAG-EnR, and CAG-Brn2-DBD-EnR are shown. Retinas were harvested at P14-P21. (A)-(C), (G)-(I), (M)-(O), CAG-EnR transfection. (D)-(F), (J)-(L), (P)-(R), CAG-Brn2-DBD-EnR transfection. (B), (E), (H), (K), (M), (Q), UB-GFP transfection. (C), (F), (I), (L), (O), (R), Merged images, tdTomato fluorescence (red), GFP fluorescence (green), DAPI staining (blue). (A), (D), 200-bp Grm6-SV40 promoter-tdTomato transfection. (G), (J), 445-bp Cabp5-tdTomato transfection. (M), (P), 164-bp Chx10-SV40 promoter-tdTomato transfection. Circles: UB-GFP-transfected bipolar cells. Scale bar: 100 μm.

FIGS. 10A-10M′″ depict gene expression in Otx2 and Crx loss-of-function mutant retinas. RNA in situ hybridization patterns from representative sections of P14 mouse retinas are shown. (A)-(M), wild-type retinal sections. (A′)-(M′), Otx2^{±} retinal sections. (A″)-(M″), Crx^{-/-} retinal sections. (A′″)-(M′″), Otx2^{±}; Crx^{-/-} retinal sections. (A)-(A′″), Grm6. (B)-(B′″), Cabp5. (C)-(C′″), Chx10. (D)-(D′″), Prkca. (E)-(E′″), Og9x. (F)-(F′″), Car8. (G)-(G′″), Nfasc. (H)-(H′″), Pcp2. (I)-(I′″), Trpm1. (J)-(J′″), 2300002D11Rik. (K)-(K′″), Scgn. (L)-(L′″), 6330514A18Rik. (M)-(M′″), Lhx3. Scale bar: 100 μm.

FIGS. 11A-11F depict assays of Chx10 CRE activity in embryonic retinas. Representative sections from in vitro embryonic day (E)11.5 mouse retinal transfections are shown. Retinas were harvested after 2 days of culture. (A) SV40 promoter-tdTomato-IRES-AP transfection. (D) 164-bp Chx10-SV40 promoter-tdTomato transfection. (B), (E), UB-GFP co-transfection. (C), (F), Merged images, tdTomato fluorescence (red), GFP fluorescence (green), DAPI staining (blue). Scale bar: 100 μm.

FIGS. 12A-12B schematically depict photoreception driving retinal activity in wild-type retinas versus rd retinas expressing ChR2 in ON bipolar cells. (A) In wild-type retinas, rhodopsin molecules in the photoreceptors initiate neural activity through different types of bipolar cells, that terminate in different depths in the IPL. (B) In rd retinas expressing ChR2 in ON bipolar cells, ChR2-mediated photoreception activates inner retinal circuitry in the absence of endogenous photoreceptors. Only the excitatory retinal neurons are shown. GCL, ganglion cell layer; IPL, inner plexiform layer; INL, inner nuclear layer; OPL, outer plexiform layer; ONL, outer nuclear layer. Choline acetyltransferase (ChAT) labels two bands in the IPL which serve as IPL depth markers. The IPL is divided into two sublaminae: the ON sublamina (light gray) contains the axon terminals of ON bipolar cells, while axon terminals of OFF bipolar cells terminate in the OFF sublamina (dark gray). The border between the ON and OFF sublaminae falls between the two ChAT bands.

FIGS. 13A-13H depict that ChR2 expression is selectively targeted to ON bipolar cells in wild-type and rd1 mouse retinas. (A) Expression cassette of the pGrm6-CY plasmid. Grm6enh, Grm6 enhancer element; pSV40, SV40 eukaryotic promoter sequence; RGpA, rabbit globin polyadenylation signal. (B)-(E), (H) Confocal images of ChR2-EYFP electroporated wild-type (B), (D) and rd1 (C), (E), (H) mouse retinas stained with anti-GFP antibodies to label ChR2-positive cells are shown. Bipolar cells are labeled in both the e-wt (B), (D) and e-rd1 (C), (E) retinas, as observed in top view (B), (C) and cross-sectional view (D), (E) projections. Note the complete absence of the outer nuclear layer in the adult rd1 retina (E). (F), (G), Percentage of ChR2-positive cells as a function of stratification depth of the axon terminals within the IPL of electroporated wild-type (F) and rd1 (G) mouse retinas. 0% depth corresponds to the proximal boundary of the IPL while 100% depth corresponds to the distal boundary; sub-0% depths indicate that axon terminals lie within the GCL. Dark green bars represent labeled rod bipolar cells while light green bars represent labeled cone bipolar cells. Dotted red lines indicate the positions of the ChAT-immunoreactive bands that serve as additional depth markers within the IPL. All labeled bipolar cell axon terminals are located in the ON sublamina. (H), A variety of ON bipolar cell types are labeled in the e-rd1 retina, based on cell morphology (bottom panels show end-on views of axon terminals) and stratification of axon terminals in the IPL. Blue, DAPI-stained cell nuclei. Scale bars, 10 μm.

FIGS. 14A-14C depict (A) a 200 bp regulatory sequence of murine Grm6 (set forth as SEQ ID NO:1; −8126 to −7927 relative to the first nucleotide of BC021919, GenBank, NIH; nucleotide positions 16160939 to 16161138 of Mus musculus chromosome 11 genomic contig with GenBank accession number NT_{—}096135.5); (B) a 445 bp regulatory sequence of murine Cabp5 (set forth as SEQ ID NO:2; −289 to +156 relative to the first nucleotide of NM_{—}013877, GenBank, NIH; nucleotide positions 10983215 to 10983659 of Mus musculus chromosome 7 genomic contig with GenBank accession number NT_{—}039413.7); and (C) a 164 bp regulatory sequence of murine Chx10 (C) (set forth as SEQ ID NO:3; −17,748 to −17,585 relative to the first nucleotide of NM_{—}007701, GenBank, NIH; nucleotide positions 43218268 to 43218431 of Mus musculus chromosome 12 genomic contig with GenBank accession number NT_{—}039551.7).

### DETAILED DESCRIPTION

The principles of the present invention may be applied with particular advantage to direct retinal cell-specific (e.g., bipolar cell-specific) expression of nucleic acid sequences and/or amino acid sequences. In certain embodiments, retinal cell-specific expression of nucleic acid sequences and/or amino acid sequences can be used to treat, prevent, reduce, inhibit and/or delay retinal cell disorders by increasing or decreasing the levels of one or more nucleic acid sequences, proteins or portions of proteins expressed in a retinal (e.g., bipolar) cell.

As used herein, the term “bipolar cell” refers to a retinal cell (e.g., a rod bipolar cell or a cone bipolar cell) that is located in the retina between photoreceptor cells and ganglion cells that most often has relatively narrow field dendrites that connect to photoreceptor and horizontal cells and relatively narrow field axon terminals that connect to amacrine and ganglion cells. Bipolar cells act to, directly or indirectly, transmit signals from the photoreceptors to the ganglion cells. There are at least ten morphologically distinct forms of bipolar cells in the mammalian retina, which includes at least nine types of cone bipolar cells and one type of rod bipolar cell. Bipolar cells can further be categorized into two different groups, ON and OFF, based on how they react to glutamate released by photoreceptor cells.

In certain exemplary embodiments, nucleic acid sequences that regulate gene expression are provided. As used herein, the term “regulatory element” refers to a nucleic acid sequence (e.g., a DNA sequence) that is responsible, at least in part, for controlling transcription of one or more associated genes. Enhancer regions and promoter regions refer to regulatory elements of DNA that function to increase and/or promote transcription, respectively, of one or more associated genes. Repressor (i.e., silencer) regions and terminator regions refer to regulatory elements of DNA that function to decrease and/or terminate transcription, respectively, of one or more associated genes. Regulatory elements and the factors that bind them are well-known in the art.

As used herein, the term “homeodomain” refers to a region of a protein and/or polypeptide that can bind to a regulatory element (e.g., a DNA sequence) and has a C-terminal recognition helix that aligns in the major groove and an unstructured amino-terminus that aligns in the minor groove. The recognition helix and the inter-helix loops are rich in arginine and lysine residues, which form hydrogen bonds with the DNA backbone. Conserved hydrophobic residues in the center of the recognition helix aid in stabilizing the helix packing. Homeodomain proteins show a preference for the DNA sequence 5′-ATTA-3′. Homeodomain binding sequences can also be identified by researchers based on the complementary strand DNA sequence, i.e., 5′-TAAT-3′

As used herein, a “POU homeodomain containing transcription factor” refers to a polypeptide and/or protein having a homeodomain and a separate, structurally homologous POU domain that contains two helix-turn-helix motifs and also binds to a regulatory element (e.g., a DNA sequence). The two domains are linked by a flexible loop that is long enough to stretch around the DNA helix, allowing the two domains to bind on opposite sides of the target DNA, collectively covering an eight-base segment closely matching the consensus sequence 5′-ATGCAAAT-3′. POU homeodomain-containing transcription factors include, but are not limited to, Pou1f1 (synonyms: GHF-1, Hmp1, Pit-1, Pit1), Pou2f1 (synonyms: Oct-1, Oct-1A, Oct-1B, Oct-1C, Oct-1z, Otf-1, Otf1), Pou2f2 (synonyms: Oct-2, Oct2a, Oct2b, Otf-2, Otf2), Pou2f3 (synonyms: Epoc-1, Oct-11a, Oct11, Otf-11, Otf11, Skin, Skin-1a, Skn-1a, Skn-1i), Pou3f1 (synonyms: Oct-6, Otf6, Scip, Test1, Tst-1, Tst1), Pou3f2 (synonyms: Brn-2, Brn2, Otf7), Pou3f3 (synonyms: Brn-1, Brn1, Otf8), Pou3f4 (synonyms: BRN-4, Brn4, Otf9), Pou4f1 (synonyms: Brn-3, Brn-3.0, Brn3, Brn3a), Pou4f2 (synonyms: Brn-3.2, Brn-3b, Brn3b, mBrn3-3R, Pou4f-rs1), Pou4f3 (synonyms: Brn-3.1, Brn3.1, Brn3c), Pou5f1 (synonyms: Oct-3, Oct-3/4, Oct-4, Oct3/4, Oct4, Otf-3, Otf-4, Otf3, Otf3-rs7, Otf3g, Otf4), Pou5f2, Pou6f1 (synonyms: cns-1, Emb), Pou6f2 (synonym: RPF-1), Unc-86 (C. elegans) and the like. As used herein, the term “POU homeodomain binding site” refers to a nucleic acid sequence (e.g., a DNA sequence) that one or more POU regions of a POU-containing polypeptide or protein binds in order to regulate transcription.

As used herein, a “paired-type homeodomain-containing transcription factor” refers to a polypeptide and/or protein having a conserved 60-amino-acid homeodomain having a conserved helix-turn-helix structure composed of three helices that binds to a regulatory element (e.g., a DNA sequence) (Ploski et al. (2004) Mol. Cell. Biol. 24:4824). Paired-type homeodomain transcription factors belong to the large category of homeodomain transcription factors that control development and differentiation (Gehring (1987) Science 236:1245).

Paired-type homeodomain transcription factors include, but are not limited to, Alx1 (synonym: Cart1), Alx3, Alx4, Arx, Crx (synonyms: Crx1), Dmbx1 (synonyms: Atx, Cdmx, Dmbx1, Mbx, Otx3), Gsc, Gsc2 (synonym: Gsc1), Hesx1 (synonym: Rpx), Mixl1 (synonyms: Mm1, Mml), Otx1, Otx2, Pax1 (synonyms: hbs, Pax-1, wavy tail, wt), Pax2 (synonym: Pax-2), Pax3 (synonym: Pax-3), Pax4 (synonym: Pax-4), Pax5 (synonyms: EBB-1, Pax-5), Pax6 (synonyms: AEY11, Dey, Gsfaey11, Pax-6), Pax7 (synonyms: Pax-7), Pax8 (synonyms: Pax-8), Pax9 (synonym: Pax-9), Phox2a (synonyms: Arix, Phox2, Phox2a, Pmx2, Pmx2a), Phox2b (synonyms: Dilp1, NBPhox, Phox2b, Pmx2b), Pitx1 (synonyms: Bft, P-OTX, Potx, Ptx1), Pitx2 (synonyms: Brx1, Brx1a, Brx1b, Munc30, Otlx2, Pitx2a, Pitx2b, Pitx2c, Ptx2, Rieg, solurshin), Pitx3 (synonym: Ptx3), Prop1 (synonyms: Prop-1, prophet of Pit-1, prophet of Pit1), Prrx1 (synonyms: mHox, mHox, Pmx1, Prx1), Prrx2 (synonym: Prx2), Rax (synonyms: ey1, Rx), Sebox (synonyms: OG9, Og9x), Shox2 (synonyms: Ogl2x, Prx3), Vsx1 (synonym: CHX10-like), Vsx2 (synonyms: Chx10, Hox-10, Hox10) and the like.

As used herein, the term “paired-type homeodomain binding site” refers to a nucleic acid sequence (e.g., a DNA sequence) that one or more paired-type homeodomain regions of a paired-type homeodomain-containing polypeptide or protein binds in order to regulate transcription.

In certain exemplary embodiments, nucleic acid sequences having one or more bipolar cell-specific regulatory elements are provided. As used herein, the term “bipolar cell-specific regulatory element” refers to a nucleic acid sequence that regulates (e.g., up-regulates or down-regulates) expression of one or more nucleic acid sequences (e.g., genes) in a bipolar cell. Bipolar cell-specific regulatory elements include nucleic acid sequences described further herein such as, e.g., the nucleic acid sequences set forth as: SEQ ID NO:1; SEQ ID NO:2; SEQ ID NO:3; nucleotides −9727 to −7113 and −76 to +409 relative to the first nucleotide of BC021919 (GenBank, NIH); nucleotides −8126 to −7113 and −76 to +409 relative to the first nucleotide of BC021919 (GenBank); nucleotides −4529 to +156 relative to the first nucleotide of NM_{—}013877 (GenBank); or nucleotides −20,102 to −17,585 relative to the first nucleotide of NM_{—}007701 (GenBank), or a portion thereof.

One aspect of the invention pertains to isolated nucleic acid molecules that encode one or more bipolar cell-specific regulatory elements. As used herein, the term “nucleic acid sequence” is intended to include DNA sequences (e.g., cDNA or genomic DNA) and RNA sequences (e.g., mRNA) and analogs of the DNA or RNA generated using nucleotide analogs. The nucleic acid sequence can be single-stranded or double-stranded, but is typically double-stranded DNA.

An “isolated” nucleic acid sequence is one which is separated from other nucleic acid sequences which are present in the natural source of the nucleic acid. In certain exemplary embodiments, an “isolated” nucleic acid sequence is free of sequences which naturally flank the nucleic acid (i.e., sequences located at the 5′ and 3′ ends of the nucleic acid) in the genomic DNA of the organism from which the nucleic acid is derived. For example, in various embodiments, the isolated, bipolar cell-specific regulatory element-containing nucleic acid sequence can contain less than about 5 kb, 4 kb, 3 kb, 2 kb, 1 kb, 0.5 kb or 0.1 kb, 100 bases, or about 10 bases of nucleotide sequences which naturally flank the nucleic acid molecule in genomic DNA of the cell from which the nucleic acid is derived. Moreover, an “isolated” nucleic acid sequence can be substantially free of other cellular material, or culture medium when produced by recombinant techniques, or substantially free of chemical precursors or other chemicals when chemically synthesized.

A nucleic acid sequence of the present invention, e.g., a nucleic acid sequence having the nucleotide sequence of SEQ ID NO:1; SEQ ID NO:2; SEQ ID NO:3; nucleotides −9727 to −7113 and −76 to +409 relative to the first nucleotide of BC021919 (GenBank, NIH); nucleotides −8126 to −7113 and −76 to +409 relative to the first nucleotide BC021919 (GenBank); nucleotides −4529 to +156 relative to the first nucleotide of NM_{—}013877 (GenBank); or nucleotides −20,102 to −17,585 relative to the first nucleotide of NM_{—}007701 (GenBank), or a portion thereof, can be isolated using standard molecular biology techniques and the sequence information provided herein. Using all or portion of the nucleic acid sequence of SEQ ID NO:1; SEQ ID NO:2; SEQ ID NO:3; nucleotides −9727 to −7113 and −76 to +409 relative to the first nucleotide of BC021919 (GenBank, NIH); nucleotides −8126 to −7113 and −76 to +409 relative to the first nucleotide of BC021919 (GenBank); nucleotides −4529 to +156 relative to the first nucleotide of NM_{—}013877 (GenBank); or nucleotides −20,102 to −17,585 relative to the first nucleotide of NM_{—}007701 (GenBank), or a portion thereof as a hybridization probe, bipolar cell-specific regulatory elements can be isolated using standard hybridization and cloning techniques (e.g., as described in Sambrook, J., Fritsh, E. F., and Maniatis, T. Molecular Cloning: A Laboratory Manual, 2nd, ed., Cold Spring Harbor Laboratory, Cold Spring Harbor Laboratory Press, Cold Spring Harbor, N.Y., 1989).

Moreover, a nucleic acid sequence encompassing all or a portion of SEQ ID NO:1; SEQ ID NO:2; SEQ ID NO:3; nucleotides −9727 to −7113 and −76 to +409 relative to the first nucleotide of BC021919 (GenBank, NIH); nucleotides −8126 to −7113 and −76 to +409 relative to the first nucleotide of BC021919 (GenBank); nucleotides −4529 to +156 relative to the first nucleotide of NM_{—}013877 (GenBank); or nucleotides −20,102 to −17,585 relative to the first nucleotide of NM_{—}007701 (GenBank), or a portion thereof, can be isolated by the polymerase chain reaction (PCR) using synthetic oligonucleotide primers designed based upon the sequence of SEQ ID NO:1; SEQ ID NO:2; SEQ ID NO:3; nucleotides −9727 to −7113 and −76 to +409 relative to the first nucleotide of BC021919 (GenBank, NIH); nucleotides −8126 to −7113 and −76 to +409 relative to the first nucleotide of BC021919 (GenBank); nucleotides −4529 to +156 relative to the first nucleotide of NM_{—}013877 (GenBank); or nucleotides −20,102 to −17,585 relative to the first nucleotide of NM_{—}007701 (GenBank).

A nucleic acid of the invention can be amplified using genomic DNA, or any combination of genomic DNA, cDNA and/or mRNA as a template and appropriate oligonucleotide primers according to standard PCR amplification techniques. For example, SEQ ID NO: 1 could be amplified from genomic DNA; SEQ ID NO:2 could be isolated from genomic DNA or a combination of genomic DNA and mRNA and/or cDNA; and SEQ ID NO:3 could be isolated from genomic DNA. The nucleic acid so amplified can be cloned into an appropriate vector and characterized by DNA sequence analysis. Furthermore, oligonucleotides corresponding to bipolar cell-specific regulatory sequences can be prepared by standard synthetic techniques, e.g., using an automated DNA synthesizer.

In certain exemplary embodiments, an isolated nucleic acid molecule of the invention comprises the nucleotide sequence shown in SEQ ID NO:1; SEQ ID NO:2; SEQ ID NO:3; nucleotides −9727 to −7113 and −76 to +409 relative to the first nucleotide of BC021919 (GenBank, NIH); nucleotides −8126 to −7113 and −76 to +409 relative to the first nucleotide of BC021919 (GenBank); nucleotides −4529 to +156 relative to the first nucleotide of NM_{—}013877 (GenBank); or nucleotides −20,102 to −17,585 relative to the first nucleotide of NM_{—}007701 (GenBank), or a portion of any of these nucleotide sequences.

In other exemplary embodiments, an isolated nucleic acid molecule of the invention comprises a nucleic acid sequence which is a complement of the nucleotide sequence shown in SEQ ID NO:1; SEQ ID NO:2; SEQ ID NO:3; nucleotides −9727 to −7113 and −76 to +409 relative to the first nucleotide of BC021919 (GenBank, NIH); nucleotides −8126 to −7113 and −76 to +409 relative to the first nucleotide of BC021919 (GenBank); nucleotides −4529 to +156 relative to the first nucleotide of NM_{—}013877 (GenBank); or nucleotides −20,102 to −17,585 relative to the first nucleotide of NM_{—}007701 (GenBank), or a portion of any of these nucleotide sequences.

A nucleic acid sequence which is complementary to the nucleotide sequence shown in SEQ ID NO:1; SEQ ID NO:2; SEQ ID NO:3; nucleotides −9727 to −7113 and −76 to +409 relative to the first nucleotide of BC021919 (GenBank, NIH); nucleotides −8126 to −7113 and −76 to +409 relative to the first nucleotide of BC021919 (GenBank); nucleotides −4529 to +156 relative to the first nucleotide of NM_{—}013877 (GenBank); or nucleotides −20,102 to −17,585 relative to the first nucleotide of NM_{—}007701 (GenBank), is one which is sufficiently complementary to the nucleotide sequence shown in SEQ ID NO:1; SEQ ID NO:2; SEQ ID NO:3; nucleotides −9727 to −7113 and −76 to +409 relative to the first nucleotide of BC021919 (GenBank, NIH); nucleotides −8126 to −7113 and −76 to +409 relative to the first nucleotide of BC021919 (GenBank); nucleotides −4529 to +156 relative to the first nucleotide of NM_{—}013877 (GenBank); or nucleotides −20,102 to −17,585 relative to the first nucleotide of NM_{—}007701 (GenBank), such that it can hybridize to the nucleotide sequence shown in SEQ ID NO:1; SEQ ID NO:2; SEQ ID NO:3; nucleotides −9727 to −7113 and −76 to +409 relative to the first nucleotide of BC021919 (GenBank, NIH); nucleotides −8126 to −7113 and −76 to +409 relative to the first nucleotide of BC021919 (GenBank); nucleotides −4529 to +156 relative to the first nucleotide of NM_{—}013877 (GenBank); or nucleotides −20,102 to −17,585 relative to the first nucleotide of NM_{—}007701 (GenBank), thereby forming a stable duplex.

In certain exemplary embodiments, an isolated nucleic acid molecule of the present invention comprises a nucleotide sequence which is at least about 50%, 55%, 60%, 65%, 70%, 75%, 80%, 85%, 90%, 95% or more identical to the entire length of the nucleotide sequence shown in SEQ ID NO:1; SEQ ID NO:2; SEQ ID NO:3; nucleotides −9727 to −7113 and −76 to +409 relative to the first nucleotide of BC021919 (GenBank, NIH); nucleotides −8126 to −7113 and −76 to +409 relative to the first nucleotide of BC021919 (GenBank); nucleotides −4529 to +156 relative to the first nucleotide of NM_{—}013877 (GenBank); or nucleotides −20,102 to −17,585 relative to the first nucleotide of NM_{—}007701 (GenBank), or a portion of any of these nucleotide sequences.

Moreover, the nucleic acid sequence of the invention can comprise only a portion of the nucleic acid sequence of SEQ ID NO:1; SEQ ID NO:2; SEQ ID NO:3; nucleotides −9727 to −7113 and −76 to +409 relative to the first nucleotide of BC021919 (GenBank, NIH); nucleotides −8126 to −7113 and −76 to +409 relative to the first nucleotide of BC021919 (GenBank); nucleotides −4529 to +156 relative to the first nucleotide of NM_{—}013877 (GenBank); or nucleotides −20,102 to −17,585 relative to the first nucleotide of NM_{—}007701 (GenBank), for example a fragment which can be used as a probe or primer or a fragment encoding a biologically active portion of a bipolar cell-specific regulatory sequence. The nucleotide sequence determined from the cloning of a bipolar cell-specific regulatory sequence allows for the generation of probes and primers designed for use in identifying and/or cloning other bipolar cell-specific regulatory sequences, as well as bipolar cell-specific regulatory sequence homologues (e.g., human, rat, dog and the like) from other species.

The probe/primer typically comprises substantially purified oligonucleotide. The oligonucleotide typically comprises a region of nucleotide sequence that hybridizes under stringent conditions to at least about 12, 15, 20, 25, 30, 35, 40, 45, 50, 55, 60, 65, 75 or more consecutive nucleotides of a sense sequence of SEQ ID NO:1; SEQ ID NO:2; SEQ ID NO:3; nucleotides −9727 to −7113 and −76 to +409 relative to the first nucleotide of BC021919 (GenBank, NIH); nucleotides −8126 to −7113 and −76 to +409 relative to the first nucleotide of BC021919 (GenBank); nucleotides −4529 to +156 relative to the first nucleotide of NM_{—}013877 (GenBank); or nucleotides −20,102 to −17,585 relative to the first nucleotide of NM_{—}007701 (GenBank), of an antisense sequence of SEQ ID NO:1; SEQ ID NO:2; SEQ ID NO:3; nucleotides −9727 to −7113 and −76 to +409 relative to the first nucleotide of BC021919 (GenBank, NIH); nucleotides −8126 to −7113 and −76 to +409 relative to the first nucleotide of BC021919 (GenBank); nucleotides −4529 to +156 relative to the first nucleotide of NM_{—}013877 (GenBank); or nucleotides −20,102 to −17,585 relative to the first nucleotide of NM_{—}007701 (GenBank), or of a naturally occurring allelic variant or mutant of SEQ ID NO:1; SEQ ID NO:2; SEQ ID NO:3; nucleotides −9727 to −7113 and −76 to +409 relative to the first nucleotide of BC021919 (GenBank, NIH); nucleotides −8126 to −7113 and −76 to +409 relative to the first nucleotide of BC021919 (GenBank); nucleotides −4529 to +156 relative to the first nucleotide of NM_{—}013877 (GenBank); or nucleotides −20,102 to −17,585 relative to the first nucleotide of NM_{—}007701 (GenBank). In an exemplary embodiment, a nucleic acid sequence of the present invention comprises a nucleotide sequence which is 150-200, 200-250, 250-300, 300-350, 350-400, 400-450, 450-500, 500-550, 550-600, 600-650, 650-700, 700-750, 750-800, 800-850, 850-900, 949, 950-1000, 1000-1500, 1500-2000, 2000-2500, 2500-3000, 3000-3500, 3500-4000, 4000-4500, 4500-5000 or more nucleotides in length and hybridizes under stringent hybridization conditions to a nucleic acid sequence of SEQ ID NO:1; SEQ ID NO:2; SEQ ID NO:3; nucleotides −9727 to −7113 and −76 to +409 relative to the first nucleotide of BC021919 (GenBank, NIH); nucleotides −8126 to −7113 and −76 to +409 relative to the first nucleotide of BC021919 (GenBank); nucleotides −4529 to +156 relative to the first nucleotide of NM_{—}013877 (GenBank); or nucleotides −20,102 to −17,585 relative to the first nucleotide of NM_{—}007701 (GenBank). In another exemplary embodiment, a nucleic acid sequence of the present invention comprises a nucleotide sequence which is approximately 164, 200, 445, 1,498, 2,517, 3,099 or 4,685 nucleotides in length and hybridizes under stringent conditions to a nucleic acid sequence of SEQ ID NO:1; SEQ ID NO:2; SEQ ID NO:3; nucleotides −9727 to −7113 and −76 to +409 relative to the first nucleotide of BC021919 (GenBank, NIH); nucleotides −8126 to −7113 and −76 to +409 relative to the first nucleotide of BC021919 (GenBank); nucleotides −4529 to +156 relative to the first nucleotide of NM_{—}013877 (GenBank); or nucleotides −20,102 to −17,585 relative to the first nucleotide of NM_{—}007701 (GenBank)

In addition to the nucleotide sequences shown in SEQ ID NO:1; SEQ ID NO:2; SEQ ID NO:3; nucleotides −9727 to −7113 and −76 to +409 relative to the first nucleotide of BC021919 (GenBank, NIH); nucleotides −8126 to −7113 and −76 to +409 relative to the first nucleotide of BC021919 (GenBank); nucleotides −4529 to +156 relative to the first nucleotide of NM_{—}013877 (GenBank); or nucleotides −20,102 to −17,585 relative to the first nucleotide of NM_{—}007701 (GenBank), it will be appreciated by those skilled in the art that DNA sequence polymorphisms may exist within a population (e.g., the human population, various mouse strains and the like). Such genetic polymorphism in one or more bipolar cell-specific regulatory sequences described herein may exist among individuals within a population due to natural allelic variation.

Nucleic acid sequences encoding one or more other bipolar cell-specific regulatory elements and which have a nucleotide sequence that differs from SEQ ID NO:1; SEQ ID NO:2; SEQ ID NO:3; nucleotides −9727 to −7113 and −76 to +409 relative to the first nucleotide of BC021919 (GenBank, NIH); nucleotides −8126 to −7113 and −76 to +409 relative to the first nucleotide of BC021919 (GenBank); nucleotides −4529 to +156 relative to the first nucleotide of NM_{—}013877 (GenBank); or nucleotides −20,102 to −17,585 relative to the first nucleotide of NM_{—}007701 (GenBank), are intended to be within the scope of the invention. For example, another (e.g., a human) bipolar cell-specific regulatory element can be identified based on the nucleotide sequence of one or more murine bipolar cell-specific regulatory elements. Moreover, nucleic acid sequences encoding one or more bipolar cell-specific regulatory elements from different species, and thus which have a nucleotide sequence which differs from the sequences of SEQ ID NO:1; SEQ ID NO:2; SEQ ID NO:3; nucleotides −9727 to −7113 and −76 to +409 relative to the first nucleotide of BC021919 (GenBank, NIH); nucleotides −8126 to −7113 and −76 to +409 relative to the first nucleotide of BC021919 (GenBank); nucleotides −4529 to +156 relative to the first nucleotide of NM_{—}013877 (GenBank); or nucleotides −20,102 to −17,585 relative to the first nucleotide of NM_{—}007701 (GenBank) are intended to be within the scope of the invention.

As used herein, the term “hybridizes under stringent conditions” is intended to describe conditions for hybridization and washing under which nucleotide sequences at least 60% identical to each other typically remain hybridized to each other. In certain exemplary embodiments, the conditions are such that sequences at least about 70%, at least about 80%, or at least about 85%, 90%, 91%, 92%, 93%, 94%, 95%, 96%, 97%, 98% identical to each other remain hybridized to each other. Such stringent conditions are known to those skilled in the art and can be found in Current Protocols in Molecular Biology, John Wiley & Sons, N.Y. (1989), 6.3.1-6.3.6. A non-limiting example of stringent hybridization conditions are hybridization in 6× sodium chloride/sodium citrate (SSC) at about 45° C., followed by one or more washes in 0.2×SSC, 0.1% SDS (e.g., at 50° C., at 55° C., at 60° C., or at 65° C.). In certain exemplary embodiments, an isolated nucleic acid molecule that hybridizes under stringent conditions to the sequence of SEQ ID NO:1; SEQ ID NO:2; SEQ ID NO:3; nucleotides −9727 to −7113 and −76 to +409 relative to the first nucleotide of BC021919 (GenBank, NIH); nucleotides −8126 to −7113 and −76 to +409 relative to the first nucleotide of BC021919 (GenBank); nucleotides −4529 to +156 relative to the first nucleotide of NM_{—}013877 (GenBank); or nucleotides −20,102 to −17,585 relative to the first nucleotide of NM_{—}007701 (GenBank), corresponds to a naturally-occurring nucleic acid sequence. As used herein, a “naturally-occurring” nucleic acid sequence refers to an RNA or DNA sequence having a nucleotide sequence that occurs in nature (e.g., can be found in a “wild-type” organism or cell).

Given the bipolar cell-specific regulatory sequences described herein, antisense nucleic acid sequences can be designed according to the rules of Watson and Crick base pairing for retinal cell-specific expression. The antisense nucleic acid sequence can be complementary to one or more retinal cell (e.g., bipolar cell) nucleic acid sequences. In certain exemplary embodiments, an antisense nucleic acid sequence is an oligonucleotide which is antisense to only a portion of one or more bipolar cell nucleic acid sequences. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine. Alternatively, the antisense nucleic acid can be produced biologically using an expression vector into which a nucleic acid has been sub-cloned in an antisense orientation.

Antisense nucleic acid sequences are typically administered to a subject or generated in situ such that they hybridize with or bind to bipolar cell mRNA and/or bipolar cell genomic DNA encoding a polypeptide to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. The antisense nucleic acid molecules can also be delivered to cells using the vectors described herein.

In yet another embodiment, one or more bipolar cell-specific regulatory element sequences described herein can be modified at the base moiety, sugar moiety or phosphate backbone to improve, e.g., the stability, hybridization, or solubility of the sequence. For example, the deoxyribose phosphate backbone of the nucleic acid molecules can be modified to generate peptide nucleic acids (see Hyrup B. et al. (1996) Bioorganic & Medicinal Chemistry 4:5). As used herein, the terms “peptide nucleic acids” or “PNA” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. Proc. Natl. Acad. Sci. USA 93:14670.

PNAs including bipolar cell-specific regulatory elements can be used in therapeutic and diagnostic applications. For example, PNAs can be used as antisense or anti-gene agents for sequence-specific modulation of bipolar gene expression by, for example, inducing transcription or translation arrest or inhibiting replication. PNAs can also be used in the analysis of single base pair mutations in a gene, (e.g., by PNA-directed PCR clamping); as ‘artificial restriction enzymes’ when used in combination with other enzymes, (e.g., S1 nucleases (Hyrup B. (1996) supra)); or as probes or primers for DNA sequencing or hybridization (Hyrup B. et al. (1996) supra; Perry-O'Keefe supra).

In another embodiment, PNAs can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. (1996) supra). The synthesis of PNA-DNA chimeras can be performed as described in Hyrup B. (1996) supra and Finn P. J. et al. (1996) Nucl. Acids Res. 24:3357. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucl. Acid Res. 17:5973). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med Chem. Lett. 5:1119).

In other embodiments, a nucleic acid sequence may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553; Lemaitre et al. (1987) Proc. Natl. Acad. Sci. USA 84:648; WO 88/09810). In addition, oligonucleotides can be modified with hybridization-triggered cleavage agents (See, e.g., Krol et al. (1988) BioTechniques 6:958) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

In certain exemplary embodiments, the levels of one or more endogenous retinal cell nucleic acid sequences and/or amino acid sequences and/or one or more heterologous nucleic acid sequences and/or amino acid sequences are expressed in an organism to treat, prevent, reduce, inhibit and/or delay one or more retinal cell disorders. The principles of the present invention may also be applied to promote and/or accelerate retinal cell loss by decreasing the levels of one or more nucleic acid sequences and/or amino acid sequences expressed in a retinal cell. In certain aspects, the present invention provides methods and materials for treating, preventing, reducing, inhibiting and/or delaying disorders and diseases associated with retinal disorders.

As used herein, the term “retinal disorder” includes, but is not limited to, disorders of the eye such as, e.g., blindness, atrophic macular degeneration, retinitis pigmentosa, iatrogenic retinopathy, retinal tears and holes, diabetic retinopathy, sickle cell retinopathy, retinal vein and artery occlusion and the like. Retinal disorders also include, but are not limited to, certain ophthalmic disorders, such as sickle cell retinopathy and retinal vein or artery occlusion.

In certain exemplary embodiments, the present invention provides methods and materials for promoting and/or accelerating bipolar cell loss to treat, prevent, inhibit, reduce and/or delay one or more disorders and/or diseases associated with aberrant retinal cell proliferation, e.g., cancer.

Cellular proliferative disorders are intended to include disorders associated with rapid proliferation. As used herein, the term “cellular proliferative disorder” includes disorders characterized by undesirable or inappropriate proliferation of one or more subset(s) of cells in a multicellular organism. The term “cancer” refers to various types of malignant neoplasms, most of which can invade surrounding tissues, and may metastasize to different sites (see, for example, PDR Medical Dictionary 1st edition (1995), incorporated herein by reference in its entirety for all purposes). The terms “neoplasm” and “tumor” refer to an abnormal tissue that grows by cellular proliferation more rapidly than normal and continues to grow after the stimuli that initiated proliferation is removed. Id. Such abnormal tissue shows partial or complete lack of structural organization and functional coordination with the normal tissue which may be either benign (i.e., benign tumor) or malignant (i.e., malignant tumor).

Examples of the types of neoplasms intended to be encompassed by the present invention include but are not limited to those neoplasms associated with cancers of retinal tissue, neural tissue, blood forming tissue, breast, skin, bone, prostate, ovaries, uterus, cervix, liver, lung, brain, larynx, gallbladder, pancreas, rectum, parathyroid, thyroid, adrenal gland, immune system, head and neck, colon, stomach, bronchi, and/or kidneys.

As used herein, the term “organism” includes, but is not limited to, a human, a non-human primate, a cow, a horse, a sheep, a goat, a pig, a dog, a cat, a rabbit, a mouse, a rat, a gerbil, a frog, a toad and a transgenic species thereof. The term “organism” further includes, but is not limited to, a yeast cell, a yeast tetrad, a yeast colony, a bacterium, a bacterial colony, a virion, virosome, virus-like particle and/or cultures thereof, and the like.

Certain aspects of the invention pertain to vectors, such as, for example, expression vectors, containing a nucleic acid encoding one or more bipolar cell-specific regulatory sequences. As used herein, the term “vector” refers to a nucleic acid sequence capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention (e.g., a nucleic acid sequence encoding one or more bipolar cell-specific regulatory sequences and/or portion(s) thereof) in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the bipolar cell-specific regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells and those which direct expression of the nucleotide sequence only in certain host cells (e.g., tissue-specific regulatory sequences).

It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or portions thereof, including fusion proteins or portions thereof, encoded by nucleic acids as described herein, in a retinal cell-specific manner.

One or more bipolar cell-specific regulatory sequences described herein can be inserted into vectors and used as gene therapy vectors. Gene therapy vectors can be delivered to a subject by, for example, intravenous injection, local administration (see, e.g., U.S. Pat. No. 5,328,470), stereotactic injection (see, e.g., Chen et al. (1994) Proc. Natl. Acad. Sci. USA 91:3054), gene gun injection, in vivo electroporation (see, e.g., Matsuda and Cepko (2007) Proc. Natl. Acad. Sci. USA 104:1027) and the like (Fynan et al. (1993) Proc. Natl. Acad. Sci. USA 90:11478). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

Any suitable virus usable for nucleic acid delivery may be used, including, but not limited to, adenovirus, adeno-associated virus, retroviruses and the like. For example, the LIA retrovirus may be used to deliver nucleic acids. The viral titer may be varied to alter the expression levels. The viral titer may be in any suitable range. For example, the viral titer can have an upper limit of about 10⁵ cfu/ml, 10⁶ cfu/m, 10⁷ cfu/ml, 10⁸ cfu/ml, 10⁹ cfu/ml, 10¹⁰ cfu/ml, 10¹¹ cfu/ml or more. The viral titer can have a lower limit of about 10¹³ cfu/ml 10¹² cfu/ml, 10¹¹ cfu/ml, 10¹⁰ cfu/ml, 10⁹ cfu/ml, 10⁸ cfu/ml, 10⁷ cfu/ml, 10⁶ cfu/ml or less. Often, the viral titer ranges from about 10⁶ cfu/ml to 10⁸ cfu/ml. More often, the range is about 10⁷ cfu/ml to 10⁸ cfu/ml. The amount of virus to be added may also be varied. The volume of virus, or other nucleic acid and reagent, added can be in any suitable range. For example the volume may have an upper limit of about 100 μl, 200 μl, 300 μl, 400 μl, 500 μl, 750 μl, 1000 μl, 1250 μl, 1500 μl or more. The volume may have a lower limit of about 1250 μl, 1000 μl, 750 μl, 500 μl, 400 μl, 300 μl 200 μl 100 μl 50 μl 25 μl or less.

Recombinant expression vectors of the invention can be designed for cell- and/or tissue-specific expression of one or more nucleotide and/or amino acid sequences in prokaryotic or eukaryotic cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

In certain exemplary embodiments, a nucleic acid described herein is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters (e.g., basal promoter sequences) are derived from polyoma, adenovirus 2, cytomegalovirus and simian virus 40 (e.g., the GL3 promoter, which is an SV40 basal promoter (Promega Corp., Madison, Wis.)). For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, one or more bipolar cell-specific regulatory elements and/or portion(s) thereof can be reproduced in bacterial cells such as E. coli, viruses such as retroviruses, insect cells, yeast or mammalian cells (such as Chinese hamster ovary cells (CHO) or COS cells). Other suitable host cells are known to those skilled in the art.

Delivery of nucleic acid sequences described herein (e.g., vector DNA) can be by any suitable method in the art. For example, delivery may be by injection, gene gun, by application of the nucleic acid in a gel, oil, or cream, by electroporation, using lipid-based transfection reagents, or by any other suitable transfection method.

As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection (e.g., using commercially available reagents such as, for example, LIPOFECTIN® (Invitrogen Corp., San Diego, Calif.), LIPOFECTAMINE® (Invitrogen), FUGENE® (Roche Applied Science, Basel, Switzerland), JETPEI™ (Polyplus-transfection Inc., New York, N.Y.), EFFECTENE® (Qiagen, Valencia, Calif.), DREAMFECT™ (OZ Biosciences, France) and the like), or electroporation (e.g., in vivo electroporation). Suitable methods for transforming or transfecting host cells can be found in Sambrook, et al. (Molecular Cloning: A Laboratory Manual. 2nd, ed., Cold Spring harbor Laboratory, Cold Spring Harbor Laboratory Press, Cold Spring Harbor, N.Y., 1989), and other laboratory manuals.

Nucleic acid molecules including one or more bipolar cell-specific regulatory elements (e.g., SEQ ID NO:1; SEQ ID NO:2; SEQ ID NO:3; nucleotides −9727 to −7113 and −76 to +409 relative to the first nucleotide of BC021919 (GenBank, NIH); nucleotides −8126 to −7113 and −76 to +409 relative to the first nucleotide of BC021919 (GenBank); nucleotides −4529 to +156 relative to the first nucleotide of NM_{—}013877 (GenBank); or nucleotides −20,102 to −17,585 relative to the first nucleotide of NM_{—}007701 (GenBank)) and/or portion(s) thereof described herein can be incorporated into pharmaceutical compositions suitable for administration. Such compositions typically comprise the nucleic acid sequence and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the nucleic acid molecules including one or more bipolar cell-specific regulatory elements and/or portion(s) thereof described herein in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: A binder such as microcrystalline cellulose, gum tragacanth or gelatin; an excipient such as starch or lactose, a disintegrating agent such as alginic, acid, Primogel, or corn starch; a lubricant such as magnesium stearate or Sterotes; a glidant: such as colloidal silicon dioxide; a sweetening agent such as sucrose or saccharin; or a flavoring agent such as peppermint, methyl salicylate, or orange flavoring.

In one embodiment, the nucleic acid sequences including one or more bipolar cell-specific regulatory elements and/or portion(s) thereof described herein are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These may be prepared according to methods known to those skilled in the art, for example, as described in U.S. Pat. No. 4,522,811.

Nasal compositions generally include nasal sprays and inhalants. Nasal sprays and inhalants can contain one or more active components and excipients such as preservatives, viscosity modifiers, emulsifiers, buffering agents and the like. Nasal sprays may be applied to the nasal cavity for local and/or systemic use. Nasal sprays may be dispensed by a non-pressurized dispenser suitable for delivery of a metered dose of the active component. Nasal inhalants are intended for delivery to the lungs by oral inhalation for local and/or systemic use. Nasal inhalants may be dispensed by a closed container system for delivery of a metered dose of one or more active components.

In one embodiment, nasal inhalants are used with an aerosol. This is accomplished by preparing an aqueous aerosol, liposomal preparation or solid particles containing the compound. A non-aqueous (e.g., fluorocarbon propellant) suspension could be used. Sonic nebulizers may be used to minimize exposing the agent to shear, which can result in degradation of the compound.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The nucleic acid molecules including one or more bipolar cell-specific regulatory elements and/or portion(s) thereof described herein can also be prepared in the form of suppositories (e.g., with conventional suppository bases such as cocoa butter and other glycerides) or retention enemas for rectal delivery.

In one embodiment, the nucleic acid molecules including one or more bipolar cell-specific regulatory elements and/or portion(s) thereof described herein are prepared with carriers that will protect them against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of the nucleic acid sequences including one or more bipolar cell-specific regulatory elements and/or portion(s) thereof described herein can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

Data obtained from cell culture assays and/or animal studies can be used in formulating a range of dosage for use in humans. The dosage typically will lie within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

One embodiment of the present invention involves a method for treatment of a retinal disorder which includes the step of administering a therapeutically effective amount of a nucleic acid sequence including one or more bipolar cell-specific regulatory elements and/or portions thereof encoding a nucleic acid sequence, a polypeptide and/or a protein which modulates, expresses, stabilizes, destabilizes, inhibits and/or activates one or more bipolar cell proteins, polypeptides and/or nucleic acid sequences to a subject. As defined herein, a therapeutically effective amount of agent (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an inhibitor can include a single treatment or, preferably, can include a series of treatments. It will also be appreciated that the effective dosage of inhibitor used for treatment may increase or decrease over the course of a particular treatment. Changes in dosage may result from the results of diagnostic assays as described herein. The pharmaceutical compositions can be included in a container, pack, or dispenser together with instructions for administration.

It is to be understood that the embodiments of the present invention which have been described are merely illustrative of some of the applications of the principles of the present invention. Numerous modifications may be made by those skilled in the art based upon the teachings presented herein without departing from the true spirit and scope of the invention. The contents of all references, patents and published patent applications cited throughout this application are hereby incorporated by reference in their entirety for all purposes.

The following examples are set forth as being representative of the present invention. These examples are not to be construed as limiting the scope of the invention as these and other equivalent embodiments will be apparent in view of the present disclosure, figures, and accompanying claims.

### EXAMPLE 1

### Grm6 CRE Isolation

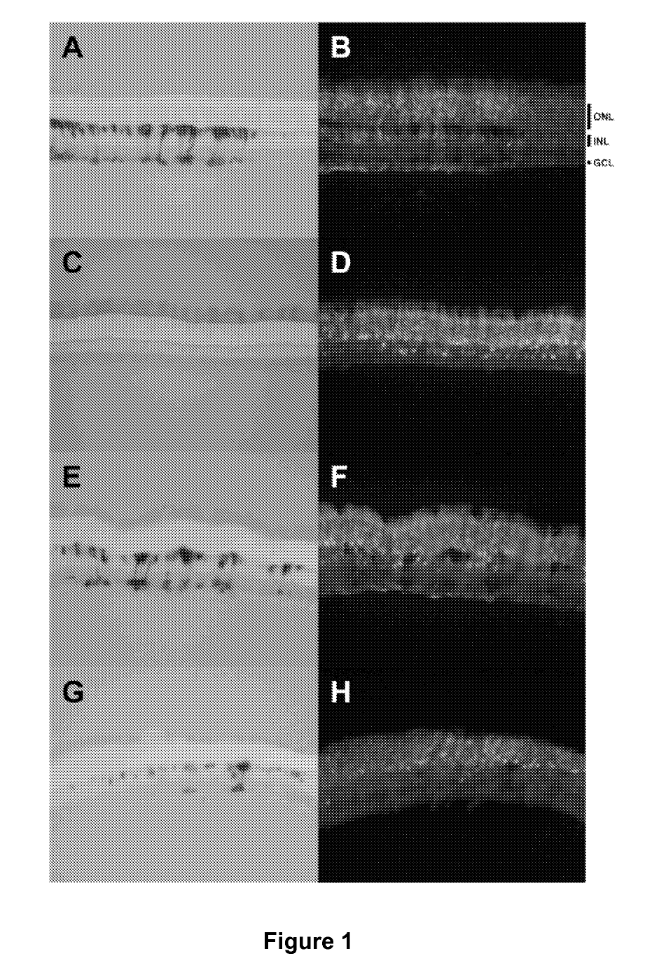

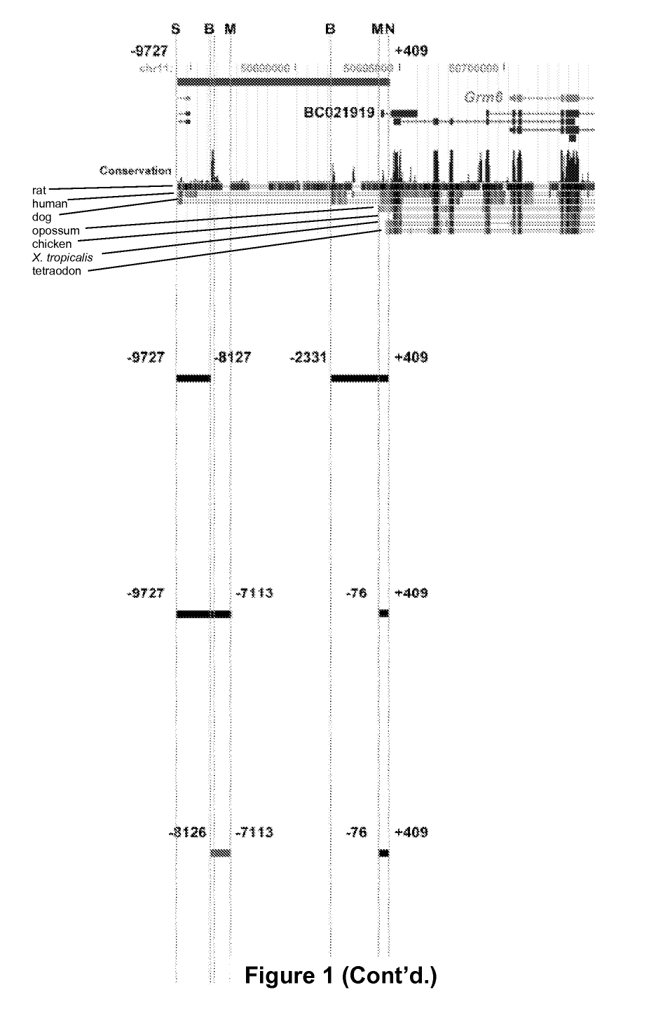

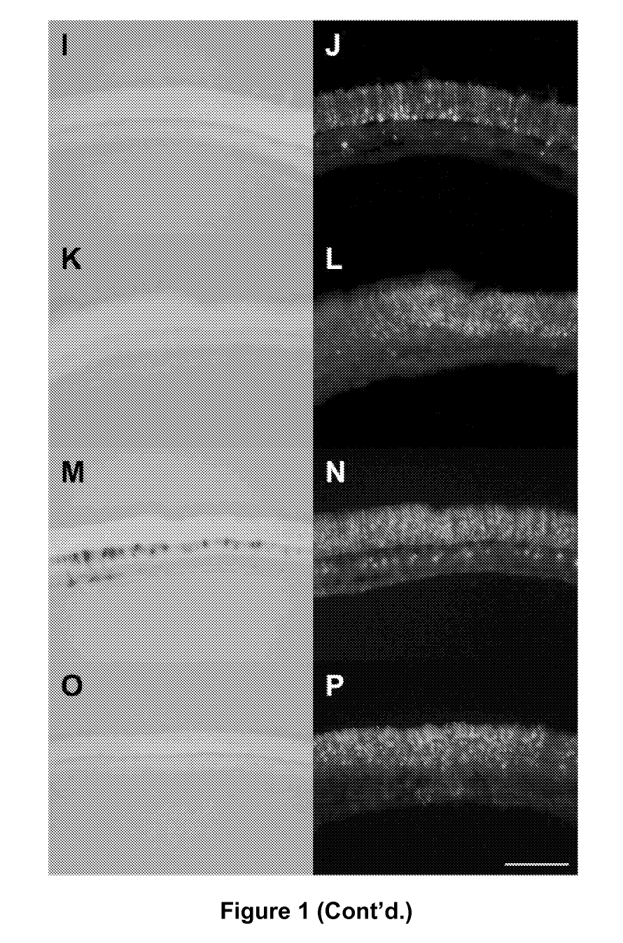

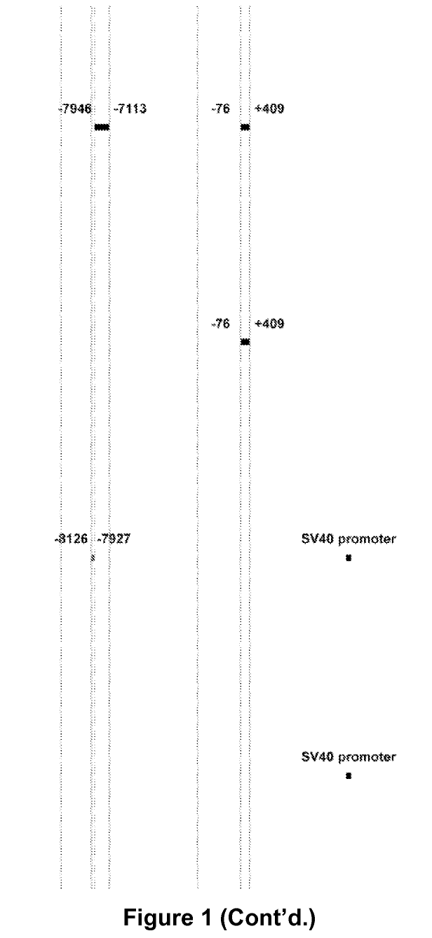

In an initial effort to characterize the CREs regulating bipolar cell genes, an analysis was conducted using in vivo retinal electroporation. A DNA construct containing a 10-kb mouse genomic fragment (−9727 to +409 relative to the first nucleotide of BC021919, GenBank, NIH) encompassing 5′ flanking sequence of the Grm6 gene that was inserted upstream of a LacZ reporter gene was transfected into neonatal rat retinas in vivo. This construct was previously shown to be sufficient to drive LacZ reporter gene expression specifically in ON bipolar cells, where Grm6 is normally expressed, in transgenic mouse lines (Vardi and Morigiwa, 1997; Nakajima et al., 1993; Ueda et al., 1997). Retinas were also co-transfected with a plasmid containing a broadly-active promoter driving GFP expression as a transfection control. Mature retinas were harvested, and histochemical staining revealed reporter gene expression specifically in ON bipolar cells, as assessed by morphological criteria. Stained cell bodies were present in the upper (scleral) part of the inner nuclear layer (INL) (FIG. 1A), and in intensely stained cells, it was possible to observe axons that projected to the lower (vitreal) half of the inner plexiform layer (IPL) where axon terminals were visible, consistent with the morphology of ON bipolar cells. GFP signal from the co-transfected plasmid was observed in many other cell types, including photoreceptor cells in the outer nuclear layer (ONL) and other INL cells (FIG. 1B), as previously reported (Matsuda and Cepko, 2004).

To determine which sequences within the original 10-kb genomic fragment were important in driving specific expression, a 5.7-kb region was removed from the construct (sequence left in construct: −9727 to −8127 and −2331 to +409). Despite expression from the co-transfected GFP plasmid in many ONL and INL cells, LacZ reporter activity was absent from transfected regions, indicating that sequences in the deleted 5.7-kb region were required to drive Grm6 expression (FIG. 1C). A different 7.0-kb region was deleted from the original 10-kb genomic sequence (sequence left in construct: −9727 to −7113 and −76 to +409). In retinas transfected with this construct, reporter expression was observed in bipolar cells, indicating that sequence in the deleted region is dispensable for Grm6 expression (FIG. 1E). Comparison of which sequences overlap in these two deletion constructs indicated that a 1-kb region (−8126 to −7113) is critical for expression. Transfection of a construct containing this 1-kb critical region and a 3′ 0.5-kb sequence (−8126 to −7113 and −76 to +409) resulted in reporter expression in bipolar cells (FIG. 1G). The 3′ 0.5-kb sequence (−76 to +409) alone was insufficient for reporter expression (FIG. 1K), confirming the importance of the 1-kb critical region in driving Grm6 transcription.

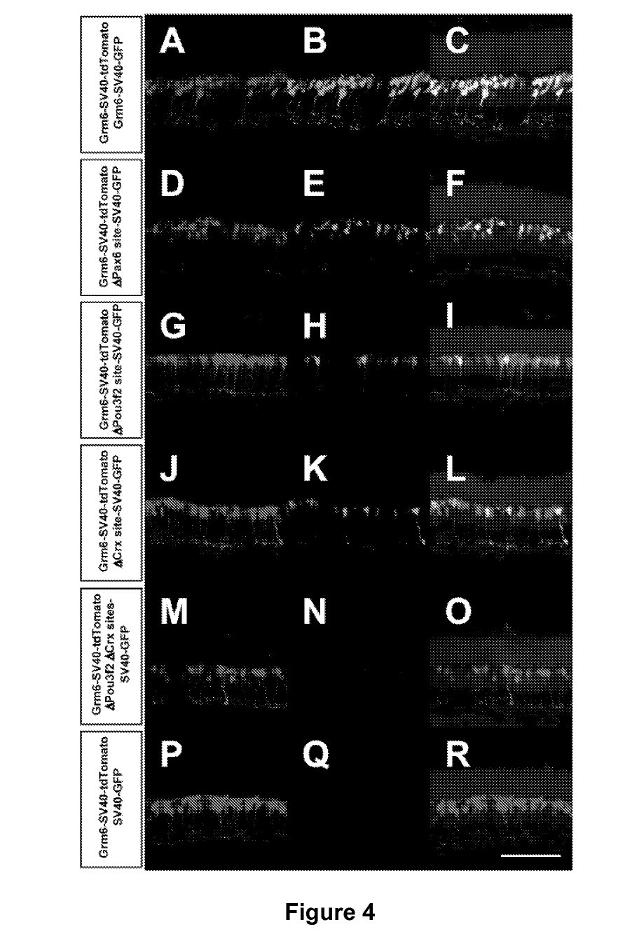

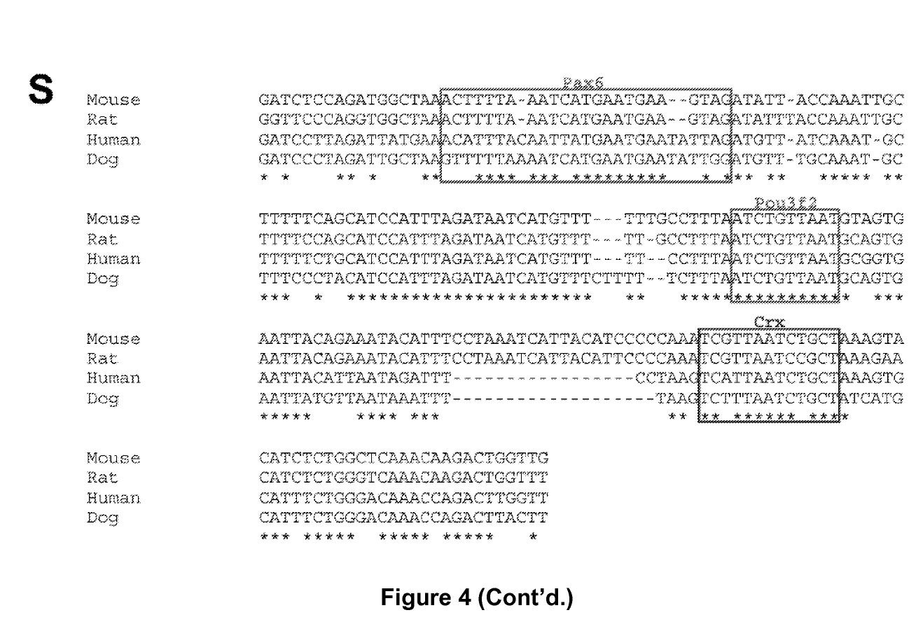

In order to further refine which sequences within this 1-kb critical region were necessary for expression and based on the hypothesis that important regulatory sequences are conserved across phylogeny, a comparison was made of this mouse sequence and syntenic sequences found in the rat, human, and dog genomes using the PhastCons program (Siepel et al., 2005). Within the critical region, only the 5′ 200-bp of the critical region (−8126 to −7927) exhibited significant conservation (FIG. 1). This mouse sequence was 94% identical with the syntenic rat sequence (FIG. 4S). Similar sequences were also found in syntenic human (78% identical) and dog (72%) genomic regions but showed lower identity. Removal of almost all of the 5′ 200-bp sequence from the construct containing the 1-kb critical region and 3′ 0.5-kb sequence (sequence in left construct: −7946 to −7113 and −76 to +409) resulted in loss of reporter expression (FIG. 1I). The 200-bp sequence, positioned 8 kb upstream of the first nucleotide (−8126 to −7927), together with a heterologous 219-bp SV40 basal promoter was sufficient to drive specific reporter expression in bipolar cells (FIG. 1M). The SV40 basal promoter alone did not exhibit detectable activity (FIG. 1O). Further fine-scale analysis of this 200-bp Grm6 CRE is discussed below.

### EXAMPLE 2

### Cabp5 CRE Isolation

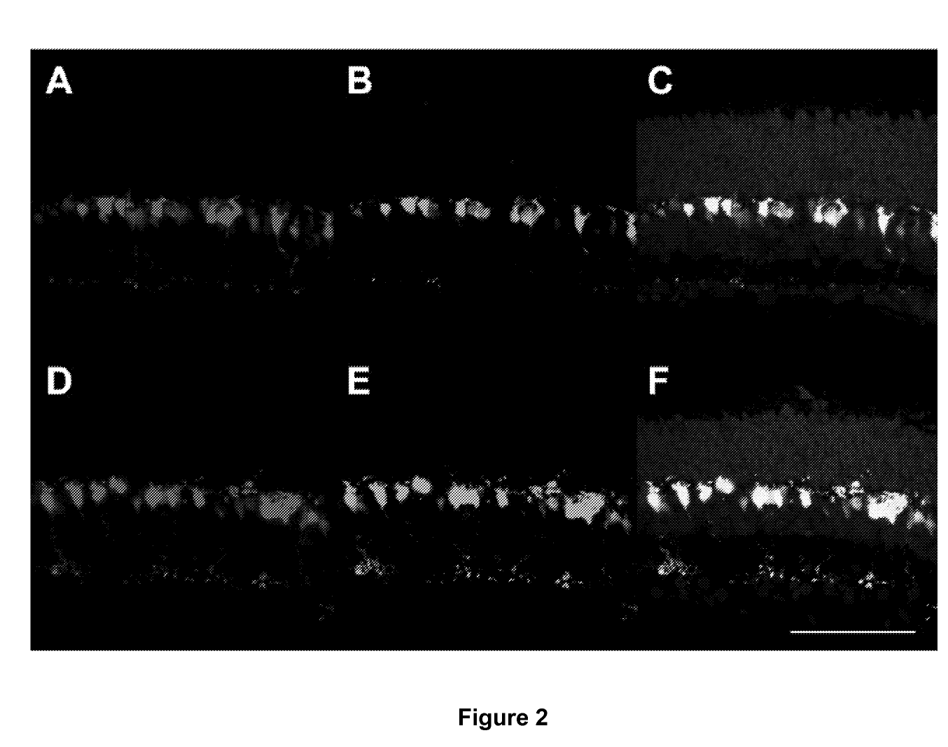

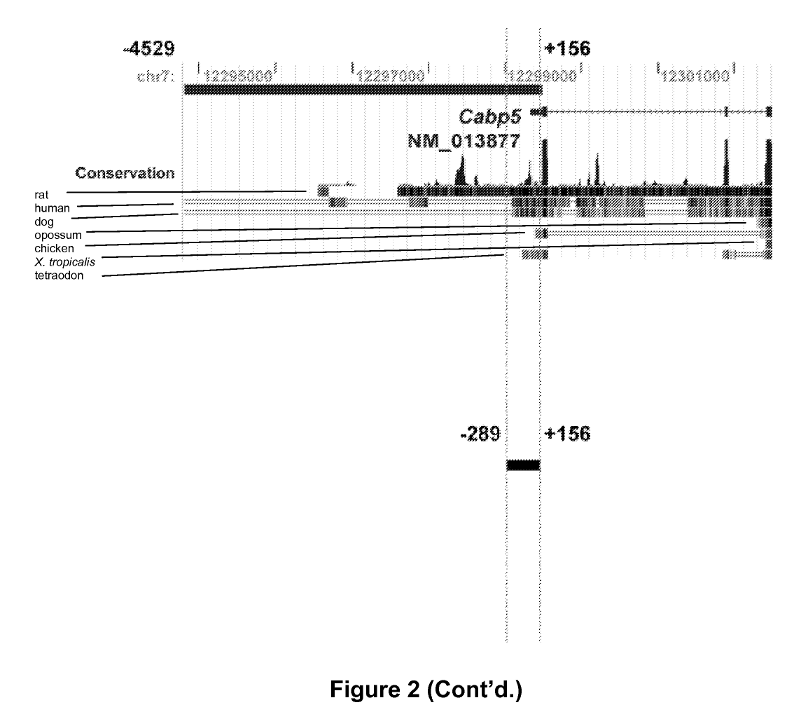

A 4.7-kb mouse genomic fragment (−4529 to +156 relative to the first nucleotide of NM_{—}013877) overlapping the 5′ untranslated region of the Cabp5 gene was inserted upstream of a GFP or tdTomato reporter construct. This genomic fragment was previously shown to direct reporter expression in a subset of bipolar cells (Matsuda and Cepko, 2004). Consistent with previous results, co-transfection of the 4.7-kb Cabp5:tdTomato and 4.7-kb Cabp5:GFP constructs into neonatal mouse retinas in vivo resulted in co-labeling of bipolar cells when mature retinas were examined (FIGS. 2A-C).

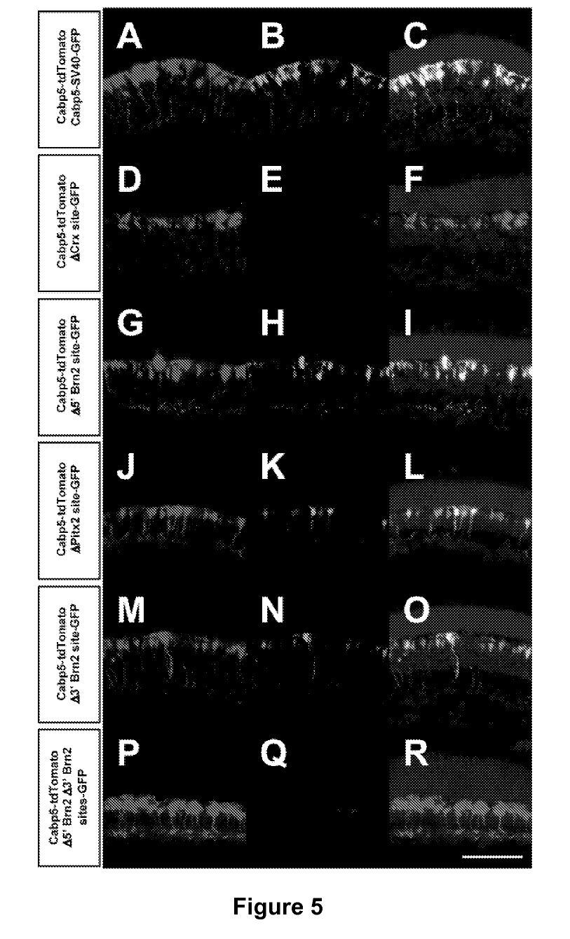

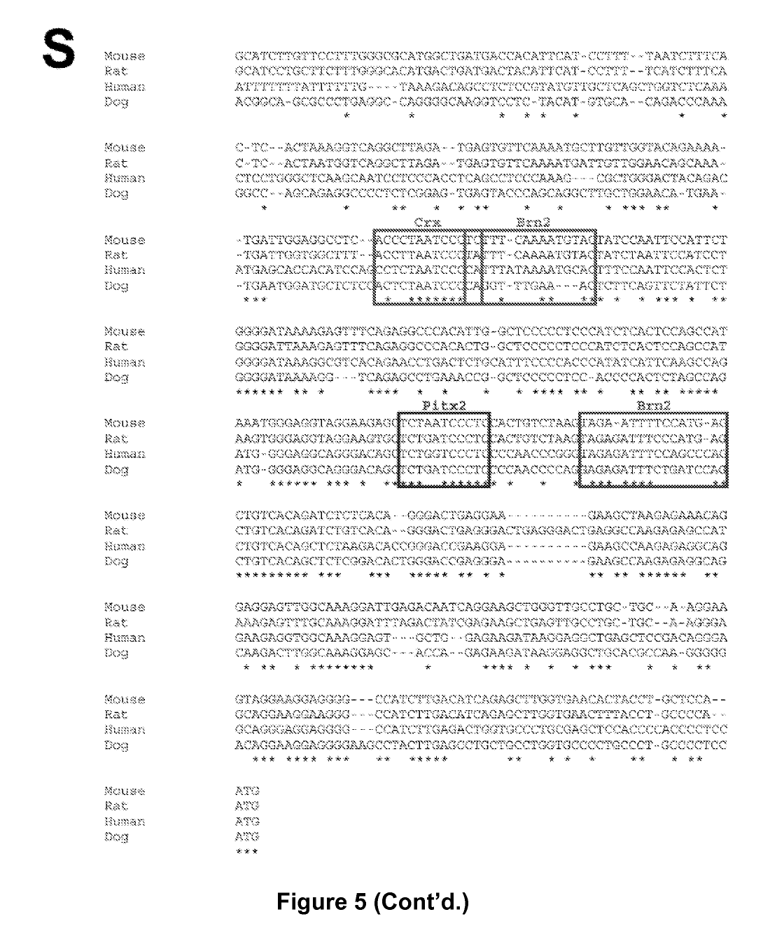

A bioinformatic comparison was made of this mouse Cabp5 flanking sequence and syntenic sequences found in the rat, human, and dog genomes. A 3′ 445-bp of the 4.7-kb sequence exhibited significant conservation across several species. This 445-bp mouse sequence was 90% identical with the syntenic rat sequence (FIG. 5S). Similar sequences were also found in syntenic human (64% identical) and dog (64%) genomic regions but showed lower identity. This 445-bp sequence (−289 to +156) was inserted upstream of a GFP reporter construct. Co-transfection of this 445-bp Cabp5-GFP construct and the 4.7-kb Cabp5:tdTomato construct resulted in co-labeling of bipolar cells, indicating that these 445 bp are sufficient to promote Cabp5 transcription (FIGS. 2D, E, F). Further fine-scale analysis of this 445-bp Cabp5 CRE is discussed below.

### EXAMPLE 3

### Chx10 CRE Isolation

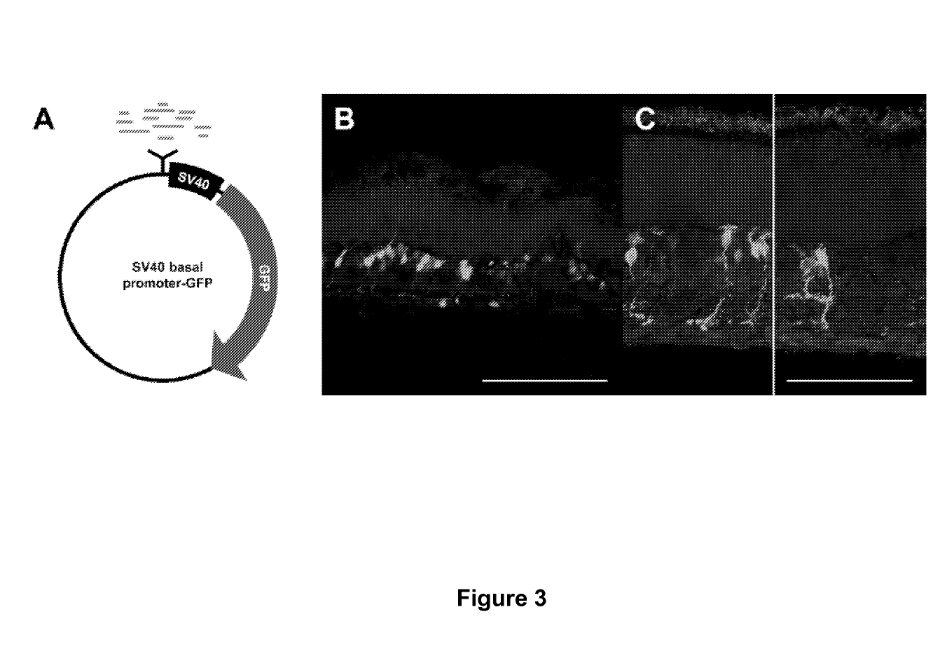

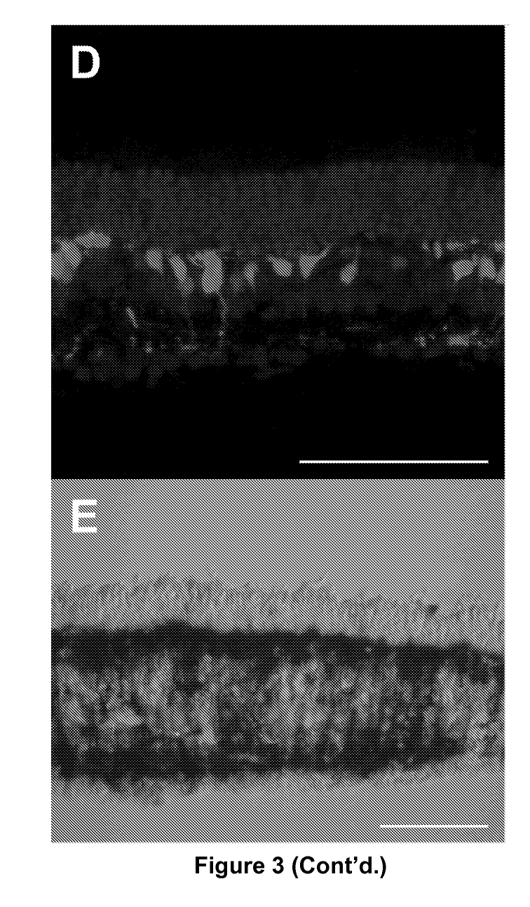

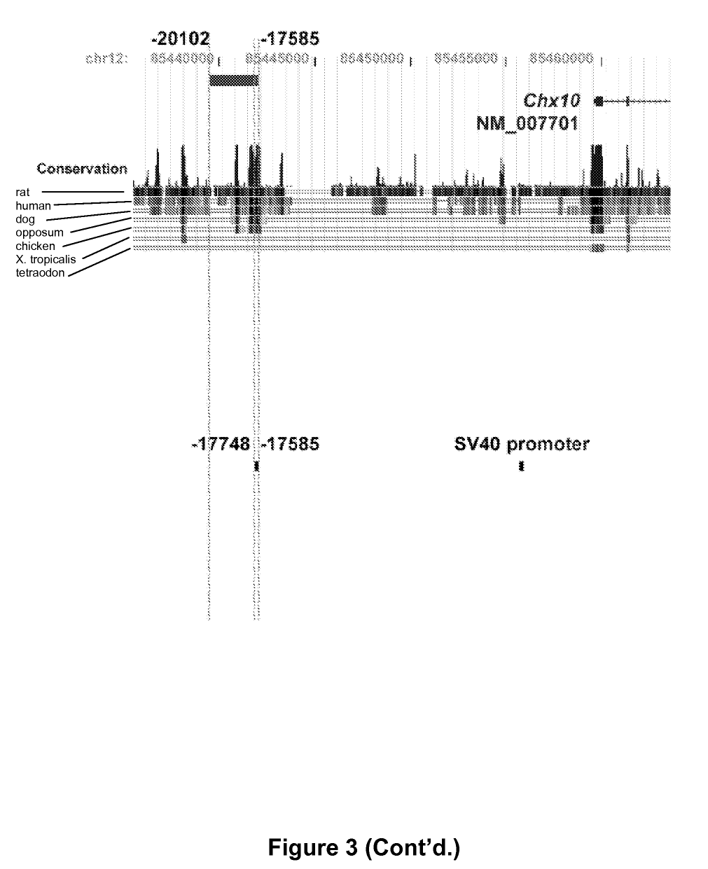

A previous study characterized a 2.4-kb Chx10 CRE overlapping the 5′ untranslated region that was sufficient to drive reporter expression in dividing progenitor cells and bipolar cells in transgenic mouse lines (Rowan and Cepko, 2005). In an effort to identify additional Chx10 regulatory elements, an unbiased screen for CREs was conducted. A 210-kb BAC containing the Chx10 gene (−109,003 to +101,164 relative to the first nucleotide of NM_{—}007701) was digested with a restriction enzyme and fragments were cloned into a reporter vector upstream of an SV40 basal promoter and a GFP reporter (FIG. 3A). Cloned fragments were tested for CRE activity by transfection by in vitro electroporation into neonatal mouse retinal explants. After 9 days of culture, retinas were examined for GFP expression. One of the twenty constructs tested was able to drive GFP expression in bipolar cells (FIG. 3B). Transfection of this construct into rat retinas by in vivo electroporation also resulted in specific reporter expression in bipolar cells, including bipolar cells projecting to the upper and lower half of the IPL, consistent with the pan-bipolar cell expression of Chx10 (FIG. 3C; Liu et al., 1994; Burmeister et al., 1996; Rowan and Cepko, 2004).

Sequencing of the insert revealed the presence of five distinct genomic fragments (−2933 to −125; −99,065 to −95,466; −20,102 to −17,589; +41,068 to +41,175; and −33,406 to −33,367) all oriented in the forward direction relative to the direction of Chx10 transcription. The middle 2.5-kb fragment (−20,102 to −17,585) situated 19 kb upstream of the Chx10 transcriptional start site together with an SV40 basal promoter was sufficient to drive expression of a fluorescent reporter construct specifically in bipolar cells when transfected into mouse retinas by in vivo electroporation (FIG. 3D). The SV40 basal promoter alone exhibited virtually no detectable background expression (FIG. 4Q).

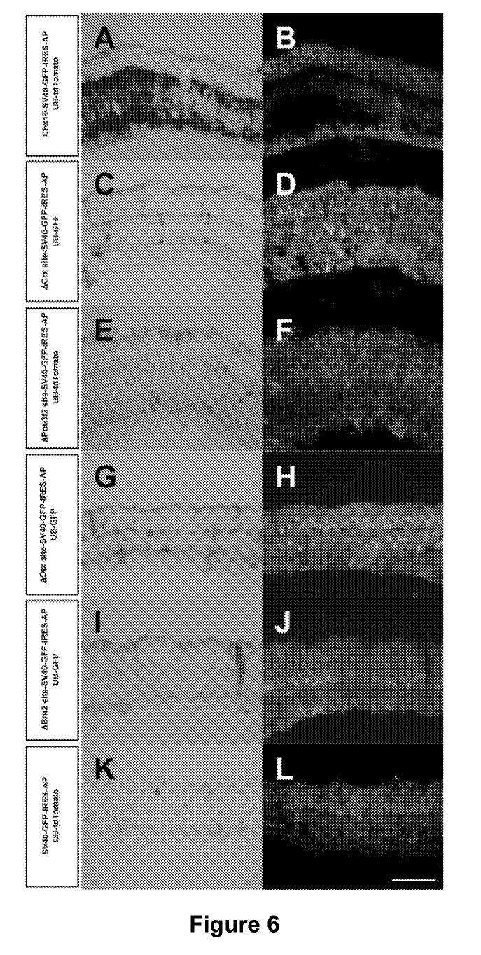

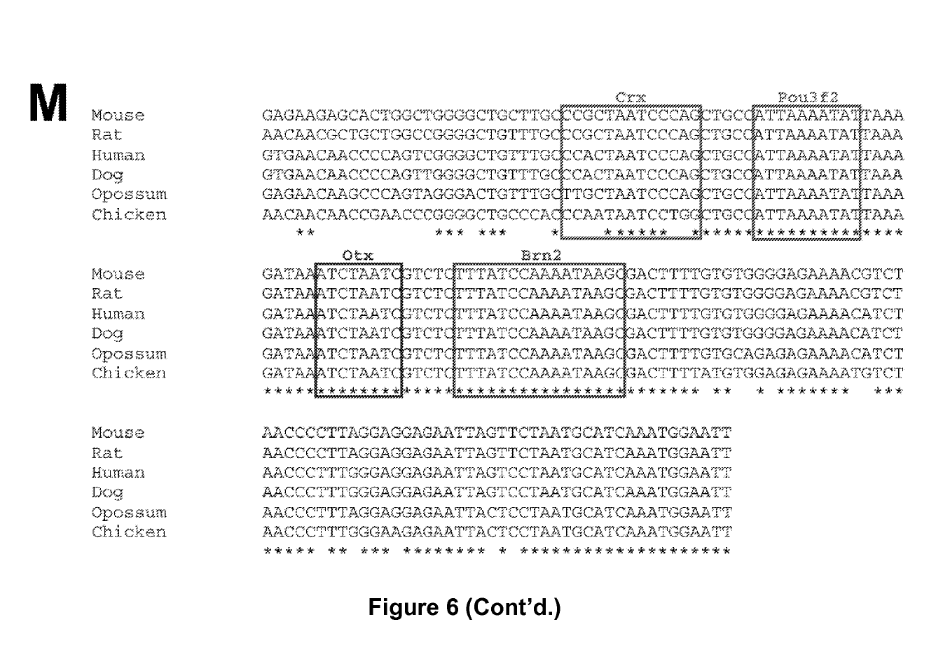

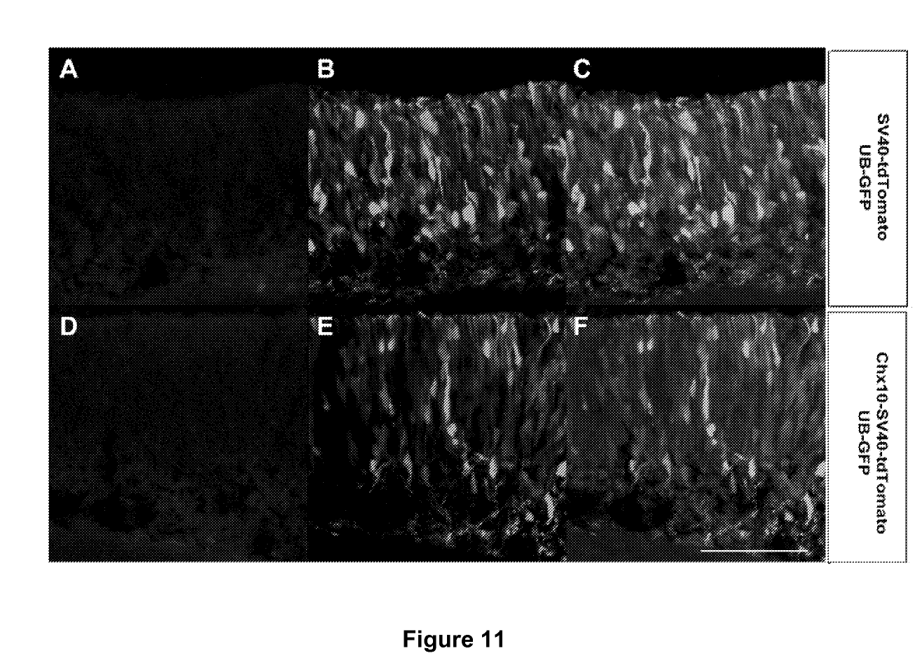

A comparison was made of this 2.5-kb Chx10 CRE sequence and syntenic sequences found in other vertebrate genomes. The 3′ 164 bp of the 2.5-kb sequence exhibited significant conservation across several species (rat—95% identical; human—91%; dog—92%; opossum—88%; chicken—85%; FIG. 6M). This 164-bp sequence (−17,748 to −17,585) together with an SV40 basal promoter was sufficient to drive specific AP reporter expression in bipolar cells (FIG. 3E). Further fine-scale analysis of this 164-bp Chx10 CRE is discussed below. Because Chx10 is normally expressed in both bipolar cells and dividing progenitor cells, the 164-bp CRE was tested for activity in embryonic retinas when many progenitor cells are present and bipolar cells are not yet present. Reporter expression was not observed following in vitro transfection of plasmids with the 164-bp sequence (−17,748 to −17,585) together with an SV40 basal promoter inserted upstream of a tdTomato construct, indicating a specific role for this CRE in Chx10 bipolar cell expression (FIG. 11).

### EXAMPLE 4

### CRE Deletion Analysis

In a further effort to characterize the transcriptional programs regulating bipolar cell genes, the 200-bp Grm6 CRE, 445-bp Cabp5 CRE, and 164-bp Chx10 CRE sequences were subjected to bioinformatic analysis to identify putative transcription factor binding sequences conserved in genomes of several species. This was done using the rVista program (version 2.0; Loots and Ovcharenko, 2004) to filter sequences through the TRANSFAC database (version 10.2; Matys et al., 2006) of 467 vertebrate transcription factor binding sequences. Each of the three regulatory elements had putative binding sites for paired-type and POU homeodomain-containing transcription factors. The 200-bp Grm6 CRE contained conserved sequences matching the Pax6, Pou3f2, and Crx TFBS matrices annotated in the TRANSFAC database (FIG. 4S). By contrast, the 445-bp Cabp5 CRE contained marginally conserved sequences matching the Crx, Brn2, and Pitx2 transcription factor binding sequence matrices (FIG. 5S). Finally, the 164-bp Chx10 CRE contained highly conserved sequences matching the Crx, Pou3f2, Otx, and Brn2 transcription factor binding sequence matrices (FIG. 6M).

The occurrence of putative paired-type and POU homeodomain-containing transcription factor binding sequences in each of the regulatory sequences indicated that these elements might be important for expression of these bipolar cell genes. To address this possibility, deletions of each of these sites were made individually and in various combinations and reporter constructs were transfected into mouse retinas in vivo. In a positive control experiment, co-transfection of plasmids containing the 200-bp Grm6 CRE and an SV40 basal promoter inserted upstream of either a tdTomato construct or a GFP construct resulted in a high incidence of co-labeling of bipolar cells (FIGS. 4A, B, C). This tdTomato-containing plasmid was then co-transfected as a control together with deletion constructs inserted upstream of an SV40 basal promoter and GFP. Fluorescent reporters were used to facilitate assessment of co-expression. Constructs containing the 200-bp CRE with the putative Pax6 site (acttttaaatcatgaatgaagtag (SEQ ID NO:44)) deleted were still able to drive GFP reporter expression in bipolar cells (FIGS. 4D, E, F). Deletion of the putative Pou3f2 site (ctgttaatgt (SEQ ID NO:47)) resulted in a decrease of GFP fluorescence in bipolar cells with only the most strongly transfected cells exhibiting GFP expression (FIGS. 4G, H, I). Deletion of the putative Crx site (cgttaatctgcta (SEQ ID NO:48)) also resulted in a diminution of GFP signal from bipolar cells (FIGS. 4J, K, L). Deletion of both the putative Pou3f2 and Crx sites led to an even greater reduction of GFP fluorescence in bipolar cells (FIGS. 4M, N, O). The putative Pou3f2 and Crx sites are thus important in activating Grm6 reporter expression. The SV40 basal promoter alone exhibited virtually no detectable background GFP expression (FIG. 4Q).

The putative Crx, Bm2, and Pitx2 sites identified in the Cabp5 CRE were tested in a similar manner. The 445-bp Cabp5 CRE inserted upstream of a tdTomato construct was used as a positive control, and deletion constructs were inserted upstream of GFP. Deletion of the putative Crx site (ccctaatccctct (SEQ ID NO:4)) resulted in a marked reduction of GFP signal from bipolar cells (FIGS. 5D, E, F). Deletion of the 5′ putative Brn2 site (tctttcaaaatgtact (SEQ ID NO:5); FIGS. 5G, H, I), the putative Pitx2 site (ctctaatccctcc (SEQ ID NO:6); FIGS. 5J, K, L), or the 3′ putative Bm2 site (agaattttccatgagc (SEQ ID NO:7); FIGS. 5M, N, O) led to a slight diminution of GFP fluorescence in bipolar cells. Deletion of both the 5′ putative Brn2 site and 3′ putative Bm2 site resulted in a near total loss of GFP signal from biopolar cells (FIGS. 5P, Q, R). The putative Crx site alone and the putative Brn2 sites together are thus critical for driving Cabp5 reporter expression.

To test the importance of putative transcription factor binding sequences in the 164-bp Chx10 CRE, deletion constructs were co-transfected with a plasmid containing a broadly-active promoter driving GFP or tdTomato as a transfection control. Deletion of the putative Crx site (ccgctaatcccag (SEQ ID NO:8)) resulted in reduction of AP reporter-positive bipolar cells (FIG. 6C). Some rod photoreceptor and cone OFF bipolar cells projecting to the upper half of the IPL were visible, indicating that this site is weakly repressive in rod photoreceptor cells and is not absolutely required for expression in cone OFF bipolar cells. Deletion of the putative Pou3f2 site (ttaaaatatt (SEQ ID NO:9)) led to near complete loss of reporter-positive cells (FIG. 6E). Deletion of the putative Otx site (ctaatcgt (SEQ ID NO:10)) resulted in reduction of reporter-positive bipolar cells (FIG. 6G). Some rod photoreceptor and cone OFF bipolar cells projecting to the upper half of the IPL were observed, indicating that this site is weakly repressive in rod photoreceptor cells and is not absolutely required for expression in cone OFF bipolar cells. Deletion of the putative Bm2 site (ttatccaaaataagcg (SEQ ID NO:11)) led to reduction of reporter-positive cells (FIG. 6I). Some Müller glial cells were visible, indicating that this site is weakly repressive in Müller glial cells. The putative Crx, Pou3f2, Otx, and Brn2 sites are thus each important in activating Chx10 transcription in bipolar cells.

### EXAMPLE 5

### Characterization of Protein Interactions with Putative Binding Sites

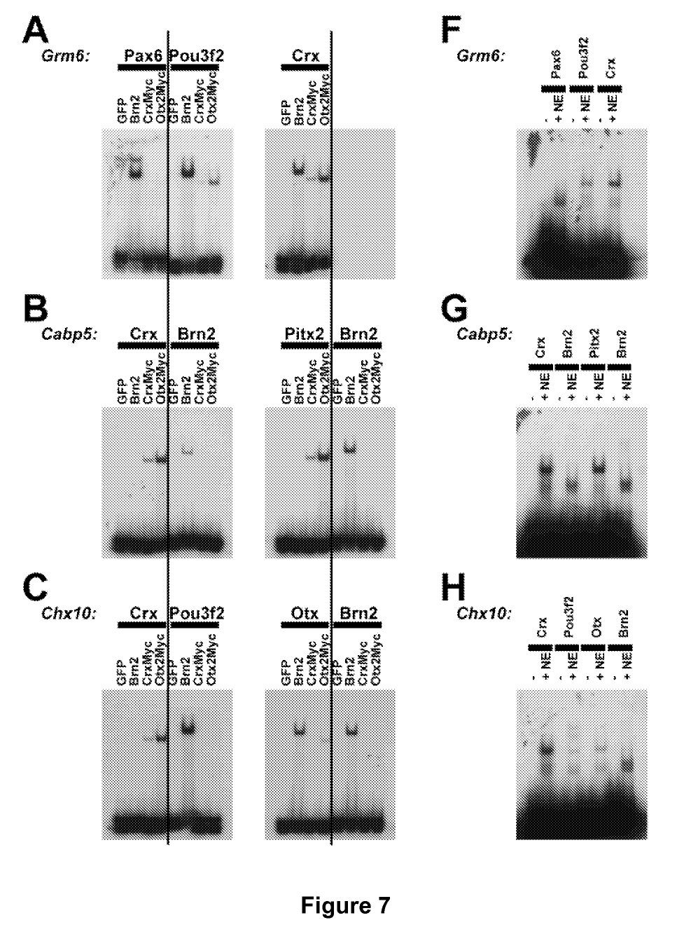

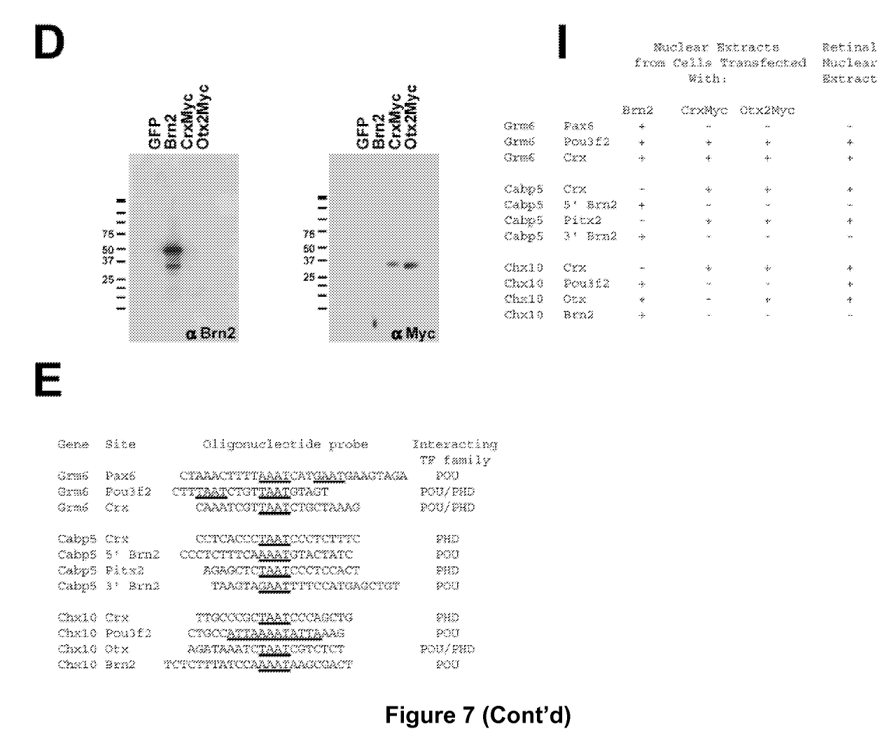

To investigate whether POU and paired-type homeodomain-containing transcription factors can interact in vitro with putative binding site sequences, electrophoretic mobility shift assays (EMSAs) were conducted using nuclear extracts from transfected 293T cells and from adult mouse retinas. Nuclear extracts from cells transfected with Bm2, Crx, and Otx2 were used for binding experiments because these POU and paired-type homeodomain-containing transcription factors have been shown to be expressed in developing and mature bipolar cells, among other retinal cell types (Rowan and Cepko, 2005; Chen et al., 1997; Furukawa et al., 1997; Koike et al., 2007). Binding activity in nuclear extracts from cells transfected with a Bm2 expression construct interacted with double-stranded oligonucleotides overlapping the putative Pax6 site in the Grm6 CRE (FIG. 7A). Binding activity was not observed when nuclear extracts from cells transfected with a GFP, myc-tagged Crx (CrxMyc), or myc-tagged Otx2 (Otx2Myc) expression construct was used, indicating that Brn2 can interact with this Pax6 site with some degree of selectivity. Nuclear extracts from cells transfected with Bm2, CrxMyc, and Otx2Myc each contained binding activity that could interact with oligonucleotides containing the putative Pou3f2 site or the putative Crx site, indicating that Bm2, Crx, and Otx2 can all interact with the Pou3f2 or Crx site (FIG. 7A). The Bm2 binding activity was of greater molecular weight than that of the CrxMyc and Otx2Myc binding activities, consistent with the relative differences in predicted molecular weights of the Bm2 (47 kD), CrxMyc (34 kD), and Otx2Myc (33 kD) proteins (He et al., 1989; Furukawa et al., 1997). Most bands appeared as singlets following gel electrophoresis, but the binding activity in nuclear extracts from cells transfected with the Brn2 was sometimes present as a doublet, which might reflect Brn2 binding to some tested sites as an oligomer or complexed with other proteins. Western blotting showed that only nuclear extract from cells transfected with Brn2 exhibited immunoreactivity with an anti-Brn2 antibody (FIG. 7D). A major band of approximately 47 kD and several lower bands, perhaps (without intending to be bound by scientific theory) degradation products, were observed. A similar experiment demonstrated that only nuclear extracts from cells transfected with CrxMyc and Otx2Myc showed immunoreactivity with an anti-Myc antibody (FIG. 7D). CrxMyc and Otx2Myc bands were ˜34 and ˜33 kD, respectively. Taken together, the data indicate that the 200-bp Grm6 CRE contains one relatively specific POU homeodomain-containing TFBS and two sites that can be bound by either POU or paired-type homeodomain-containing transcription factors (FIG. 7I).

The putative binding sites found in the Cabp5 CRE were also subjected to EMSA analysis. Nuclear extracts from cells transfected with a CrxMyc or Otx2Myc construct contained binding activity that interacted with oligonucleotides containing the Crx or Pitx2 site in the Cabp5 CRE (FIG. 7B). In contrast, nuclear extract from cells transfected with a Brn2 construct contained binding activity that interacted with oligonucleotides overlapping the 5′ Brn2 or 3′ Brn2 site. The results indicate that the 445-bp Cabp5 CRE contains two paired-type homeodomain-containing TFBSs and two POU homeodomain-containing TFBSs (FIG. 7I).

The putative binding sites identified in the Chx10 CRE were also analyzed using EMSAs. Nuclear extracts from cells transfected with CrxMyc or Otx2Myc contained binding activity that could interact with oligonucleotides overlapping the Crx site in the Chx10 CRE (FIG. 7C). In contrast, nuclear extracts from cell transfected with Brn2 contained binding activity that could interact with oligonucleotides overlapping the Pou3f2 or Brn2 site. Additionally, nuclear extracts from cells transfected with Brn2 or Otx2Myc contained binding activity that could interact with oligonucleotides overlapping the Otx site. Taken together, the data indicate that the 164-bp Chx10 CRE contains one relatively specific paired-type homeodomain-containing TFBS, two relatively specific POU homeodomain-containing transcription factor binding sequences, and one site that can be bound by either POU or paired-type homeodomain-containing transcription factors (FIG. 7I).

EMSA analysis was also conducted using the same oligonucleotides overlapping identified sites in the Grm6, Cabp5, and Chx10 regulatory sequences and nuclear extracts from adult mouse retinas. Only seven of eleven sites tested interacted with binding activity in retinal nuclear extract, even though low molecular weight, non-specific bands could be observed for all probes assayed (FIGS. 7F-H). In general, there was an almost complete correlation between presence of binding activity in retinal nuclear extracts and presence of binding activity in cells transfected with CrxMyc (FIG. 7I), indicating that Crx expressed in photoreceptor cells, which are an abundant cell type in the retina (>70%; Young, 1985), could be the factor in native retinal nuclear extracts interacting with these sites. Additionally, in every case except for one putative binding site (Chx10 CRE, Pou3f2 site), when binding activity was observed only in cells transfected with Brn2 for a given site, no binding activity was detected in nuclear extract from retinas. This indicates that while Brn2 can bind to these sites when cell nuclear extract from transfected cells is used, the in vivo Brn2 expression levels could be too low for binding activity to be detected in EMSAs when nuclear extract from adult mouse retinas is used.

Alignment and comparison of transcription factor binding sequences revealed at least three types of sites with regard to binding activities in transfected cell nuclear extracts. Sites that contained AAAT or GAAT sequences interacted only with binding activities in nuclear extract from cells expressing the POU homeodomain-containing transcription factor, Brn2, in almost every case (FIG. 7E). In contrast, sites that contained the core TAAT homeodomain-binding sequence could interact with binding activities in nuclear extracts from cells transfected with paired-type homeodomain-containing transcription factors, Crx and Otx2, and sometimes also with those in Brn2-transfected cells (Laughon, 1991). Finally, sites that contained a CTAATCC sequence interacted only with binding activities in nuclear extracts from CrxMyc- and Otx2Myc-transfected cells.

### EXAMPLE 6

### Otx2 Conditional Loss-of-Function Effects on Reporter Expression

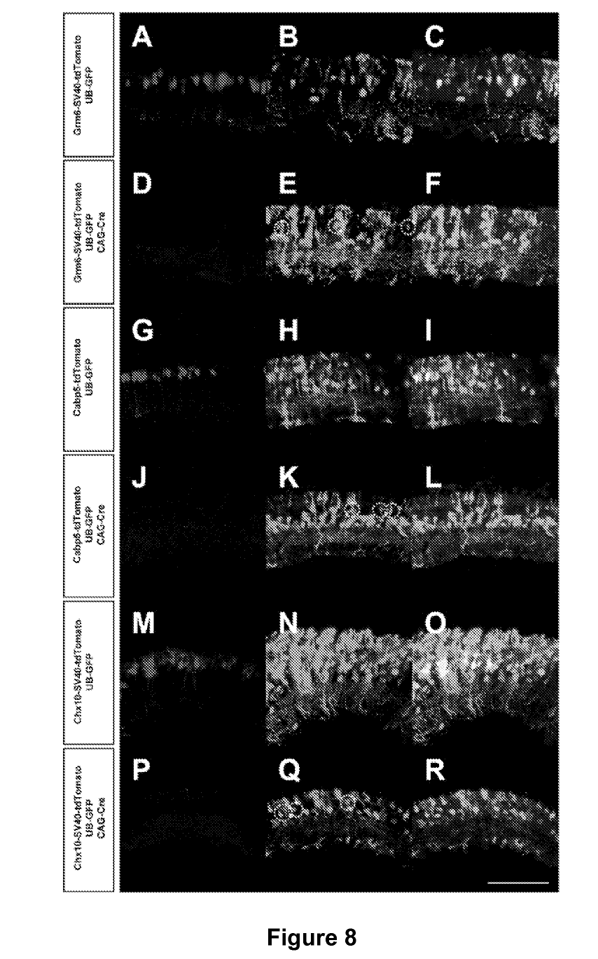

Otx2 is highly enriched in its expression in the INL, where it is found in the majority of bipolar cells of the adult retina (Koike et al., 2007). Previous studies have shown that Otx2 plays a critical role in bipolar cell terminal differentiation. To test the hypothesis that Otx2 regulates expression of Grm6, Cabp5, and Chx10, reporter expression was examined in control and Otx2 conditional loss-of-function retinas. Retinas from neonatal Otx2^{flox/flox} mice were transfected by in vivo electroporation with a plasmid containing the 200-bp Grm6 regulatory element inserted upstream of an SV40 basal promoter and a tdTomato construct as well as with a plasmid with a broadly-active promoter driving GFP. Otx2 loss-of-function was achieved by co-transfecting a subset of retinas with reporters and a plasmid containing a broadly-active promoter driving Cre recombinase expression. Co-transfected cells, indicated by GFP expression, would be expected to have undergone Cre-mediated deletion of the Otx2 gene. When mature control retinas were examined, tdTomato signal was observed in many transfected bipolar cells (FIG. 8A). But virtually no tdTomato signal could be seen in Otx2 conditional knockout (CKO) retinas, indicating that Otx2 is required for activation of this Grm6 regulatory element (FIGS. 8D, E, F). Similarly, the activity of both the 445-bp Cabp5-tdTomato (FIGS. 8J, K, L) and 164-bp Chx10-SV40-tdTomato (FIGS. 8P, Q, R) reporter constructs was attenuated in Otx2 CKO retinas, despite presence of co-transfected GFP-positive bipolar cells, indicating that Otx2 is also required for activation of the Cabp5 and Chx10 regulatory elements.

### EXAMPLE 7

### Dominant-Negative Brn2 Effects on Reporter Expression

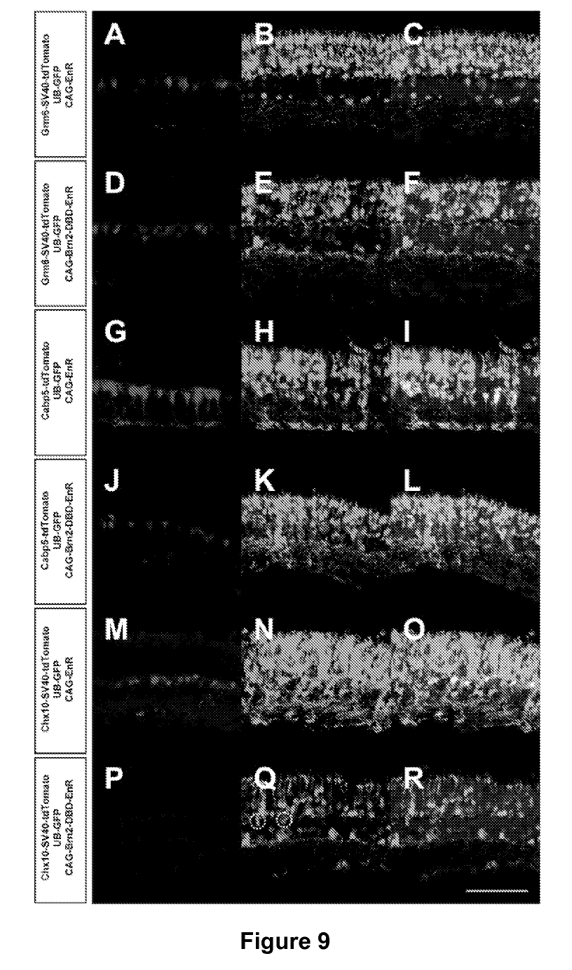

Brn2 has been demonstrated to be expressed in bipolar cells, among other cell types in the retina (Rowan and Cepko, 2005). To address the hypothesis that Brn2 is required for expression of bipolar cell genes, reporter expression was examined in retinas transfected with a dominant-negative CAG-Bm2-DBD-EnR construct containing the Brn2 DNA binding domain fused to the Drosophila engrailed transcriptional repressor domain. In control experiments, neonatal retinas were transfected with a plasmid containing a broadly-active promoter driving the engrailed repressor alone, a plasmid containing the 200-bp Grm6 CRE inserted upstream of an SV40 basal promoter and a tdTomato construct, and a plasmid with a broadly-active promoter driving GFP. Examination of mature retinas revealed tdTomato signal in many transfected bipolar cells (FIG. 9A). The tdTomato signal was similar in bipolar cells from retinas co-transfected with the CAG-Bm2-DBD-EnR construct (FIG. 9D). By contrast, the tdTomato signal was substantially reduced in bipolar cells from retinas co-transfected with the 445-bp Cabp5-tdTomato and CAG-Brn2-DBD-EnR constructs (FIGS. 9G-L). Finally, virtually no tdTomato signal was observed in retinas co-transfected with the 164-bp Chx10-SV40-tdTomato and CAG-Brn2-DBD-EnR constructs, despite presence of co-transfected GFP-positive bipolar cells (FIGS. 9M-R). The results indicate that recruitment of a transcriptional repressor to Brn2 binding sites can reduce Grm6, Cabp5, and Chx10 expression.

### EXAMPLE 8

### Otx2 and Crx Loss-of-Function Effects on Endogenous Bipolar Gene Expression

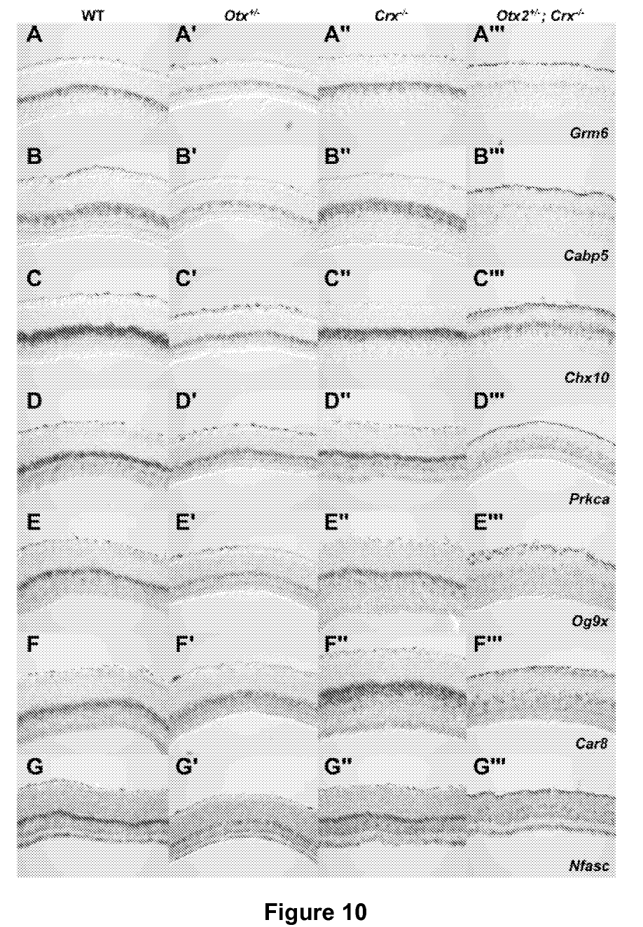

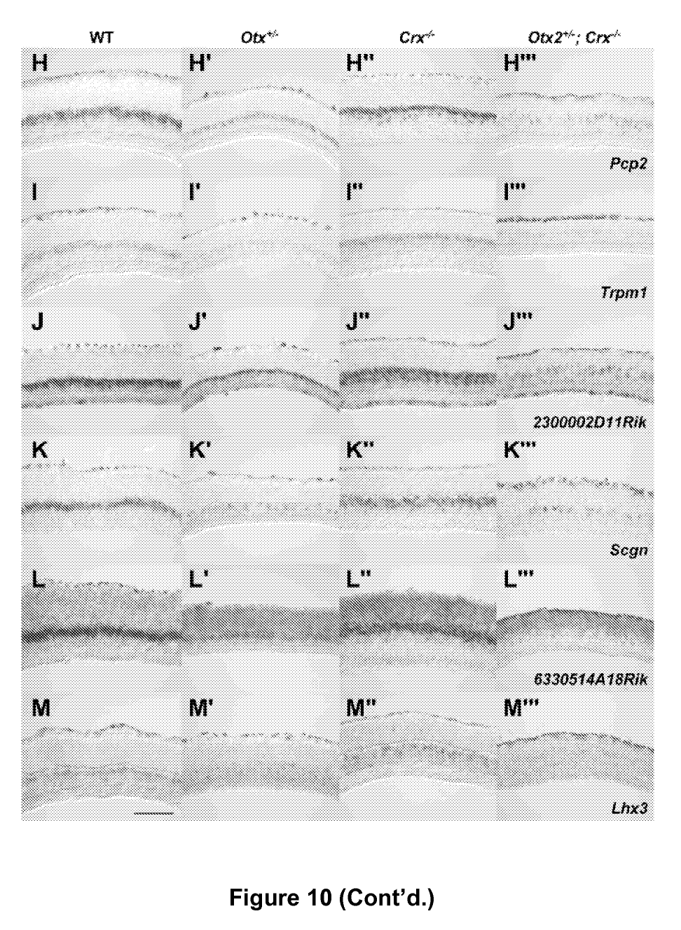

The functional requirement for the paired-type homeodomain-containing transcription factors, Otx2 and Crx, in regulating endogenous gene expression was assayed by RNA in situ hybridization. Retinal sections from wild-type, Otx2^{±}, Crx^{-/-}, and Otx2^{±}; Crx^{-/-} mice were examined at P14 prior to onset of retinopathy observed in Crx-deficient mice. Otx2-deficient mice were not examined because the forebrain neuroectoderm fails to develop in these embryos, and homozygous null mutations lead to embryonic lethality (Acampora et al., 1995). The hybridization signals for Grm6, Cabp5, and Chx10 were all attenuated in Otx2^{±} retinas (FIGS. 10A′, B′, C′). The hybridization signals for these genes were unchanged in Crx^{-/-} retinas (FIGS. 10A″, B″, C″). However, for Grm6 and Cabp5, Crx deficiency led to an even greater attenuation of hybridization signal in the Otx2^{±} background (FIGS. 10A′″, B′″, C′″). These results are consistent with a role for Otx2 by itself in activation of Grm6, Cabp5, and Chx10 transcription in bipolar cells and previously described roles for Otx2 together with Crx in bipolar cell genesis and/or survival (Koike et al., 2007; Sato et al., 2007).

To assess to extent of genes potentially regulated by Otx2 and/or Crx, additional bipolar cell markers were examined by RNA in situ hybridization, including the rod bipolar-selective makers, Prkca, Og9x, Car8, and Nfasc (FIGS. 10D-G′″); the mixed rod and cone bipolar-selective markers, Pcp2, Trpm1, and 2300002D11Rik (FIGS. 10H-J′″); and the cone bipolar cell markers, Scgn, 6330514A18Rik, and Lhx3 (FIGS. 10K-M′″; Kim et al., 2007). The hybridization signals for these genes were all attenuated in Otx2^{±} retinas. The hybridization signals for these genes were unchanged in Crx^{-/-} retinas. Crx deficiency led to an even greater attenuation of hybridization signal in the Otx2^{±} background.

### EXAMPLE 9

### Targeting Expression of ChR2 Exclusively to Retinal ON Bipolar Cells

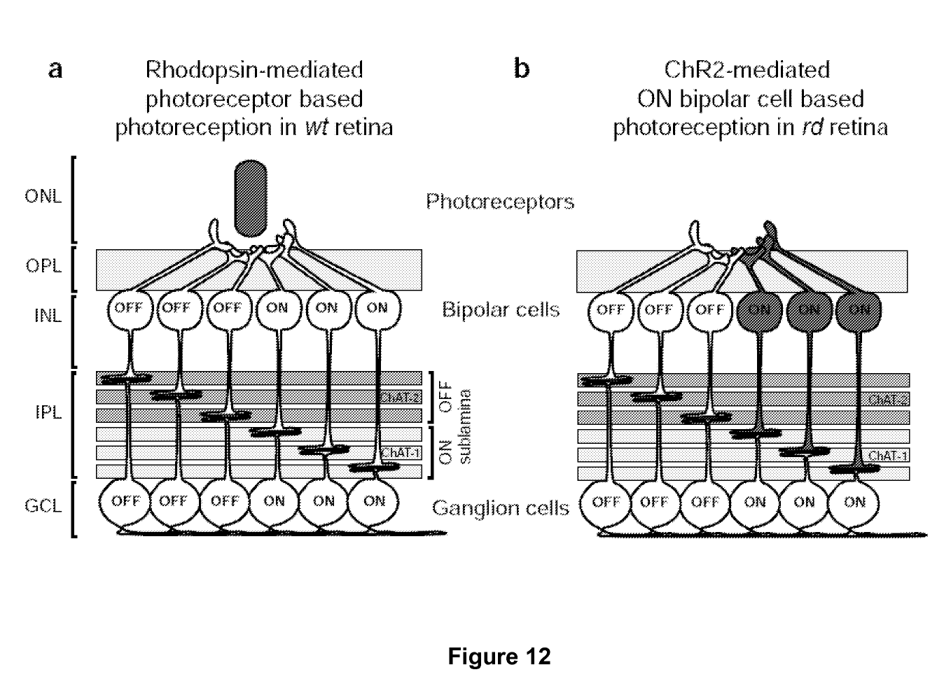

ChR2 was genetically targeted to retinal ON bipolar cells (FIG. 12) using a 200-base pair promoter sequence of the mouse Grm6 gene (SEQ ID NO: 1) which encodes the ON bipolar cell-specific metabotropic glutamate receptor, mGluR6 (Masu et al. (1995) Cell 80:757). ChR2 is a light-gated cation channel originally isolated from the green algae, Chlamydomonas reinhardtii (Nagel et al. (2003) Proc. Natl. Acad. Sci. USA 100:13940). Illumination of ChR2 leads to depolarization and therefore functional activation of neurons that express the channel (Bi et al. (2006) Neuron 50:23; Nagel et al., supra; Boyden et al. (2005) Nat. Neurosci. 8:1263).

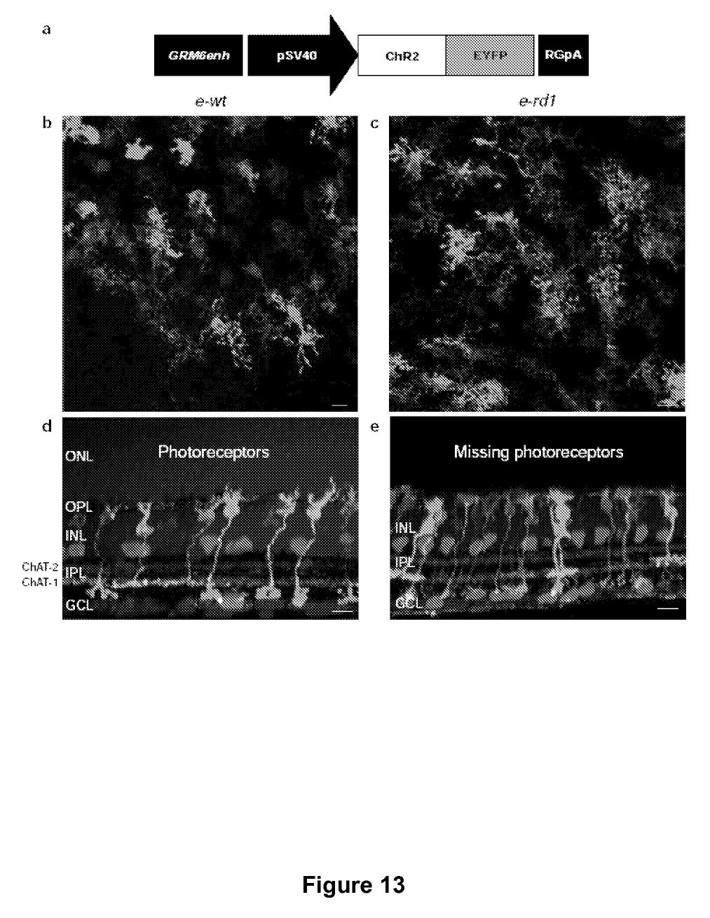

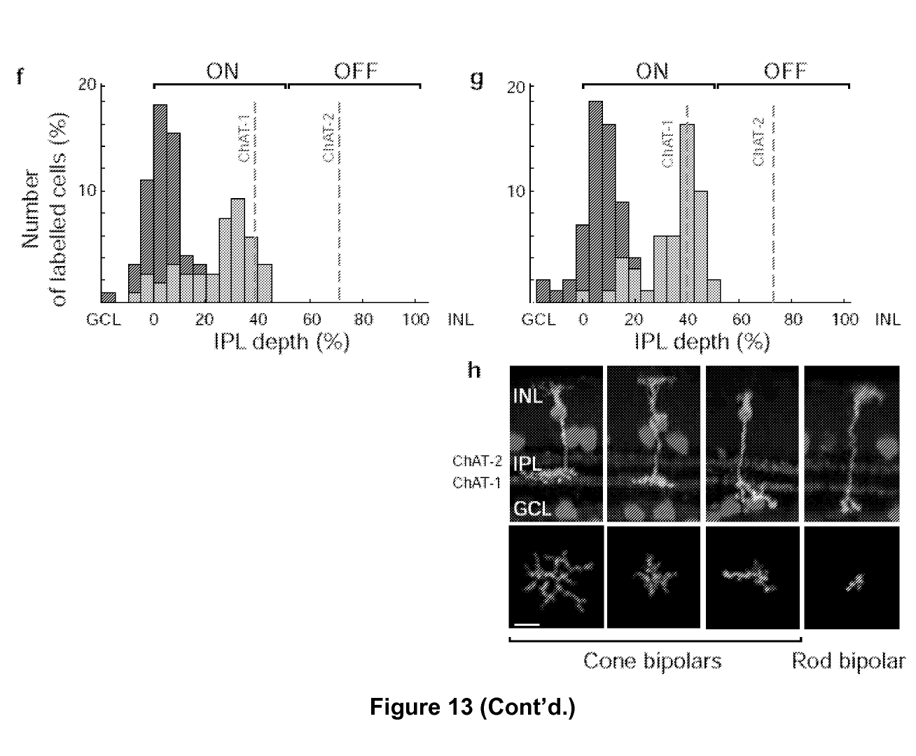

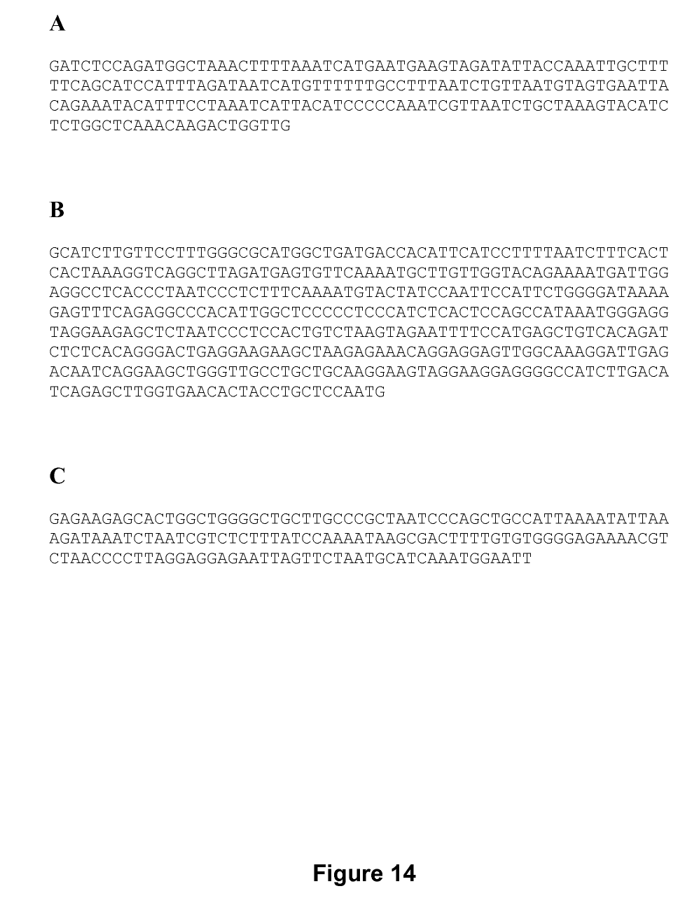

ChR2 was delivered to the retinas of wild-type (wt) mice and those of the rd1 mouse model of retinal degeneration, in which most photoreceptors are lost and the electroretinogram (ERG) is undetectable by 4 weeks of age (Farber et al. (1994) Prog. Retin. Eye Res. 13:31). In vivo electroporation (Matsuda and Cepko (2004) Proc. Natl. Acad. Sci. USA 101:16) of a Grm6 enhancer-driven ChR2-EYFP fusion construct (FIG. 13A) labeled exclusively ON bipolar cells in both wt and rd1 retinas (FIGS. 13B-13G). These cells were identified by two criteria: the cells were bipolar neurons and their axon terminals ended in the proximal part of the inner plexiform layer (IPL), known as the ON sublamina, a characteristic of ON bipolar cells in the mammalian retina (Wassle (2004) Nat. Rev. Neurosci. 5:747) (FIG. 12, FIGS. 13D-13E). EYFP expression was localized to the plasma membrane, efficiently labeling cell somas, dendritic arbors, axons and axon terminals. 7±3% of all ON bipolar cells (Jeon et al. (1998) J. Neurosci. 18:8936) were labeled in electroporated areas (4.0±2.6 mm , n=13). Expression was stable for at least 6 months post-injection in both wt and rd1 retinas. Rd1 mice that were electroporated with the ChR2 construct are designated as “e-rd1,” and electroporated, wild-type mice are designated as “e-wt.” “Rd-1 and “wt” refer to mice or retinas that are not electroporated. Immunoreactivity against the rod bipolar cell marker, PKCα, was observed for 50% of the EYFP-positive cells in both electroporated e-wt and e-rd1 retinas, indicating that approximately equal numbers of rod and cone bipolar cells expressed the ChR2-EYFP fusion protein. Axon terminals of EYFP-positive cone bipolar cells were layered in different depths in the IPL, indicating that different subtypes were labeled (FIGS. 13F-13H). These results indicate that ChR2 can be efficiently targeted to different subtypes of ON bipolar cells using in vivo electroporation in both wt and rd1 mice. ChR2 expression and activation in retinal ON bipolar cells generated excitatory light responses in ON retinal ganglion cells and inhibitory responses in OFF retinal ganglion cells. ChR2 delivery to and activity in retinal ON bipolar cells could restore photo-responsiveness to rd1 retinas.

Plasmid DNA Construction and In Vivo Electroporation

DNA constructs were generated using standard molecular biology protocols. The numbering system used in this example differs from the numbering system recited in other portions of the detailed description, figures and examples. A 200-base pair DNA sequence corresponding to nucleotide positions −8583 to −8384 (i.e., the −8126 to −7927 sequence recited elsewhere) relative to the ATG start codon of the mouse metabotropic glutamate receptor subtype 6 (Grm6) gene (Ueda et al. (1997) J. Neurosci. 17:3014) (nucleotide positions 16160939 to 16161138 of Mus musculus chromosome 11 genomic contig with GenBank accession no. NT_{—}096135) is a Grm6 enhancer element. This element was amplified from the genomic sequence upstream of the Grm6 coding region using the primers 5′-gactagtGATCTCCAGATGGCTAAAC-3′ (SEQ ID NO:45) and 5′-ccgctcgagCAACCAGTCTTGTTTGAGCC-3′ (SEQ ID NO:46) to give the 200-base pair enhancer sequence, 5′-GATCTCCAGATGGCTAAACTTTTAAATCATGAATGAAGTAGATATTAC CAAATTGCTTTTTCAGCATCCATTTAGATAATCATGTTTTTTGCCTTTA ATCTGTTAATGTAGTGAATTACAGAAATACATTTCCTAAAT CAT TACAT CCCCCAAATCGTTAATCTGCTAAAGTACATCTCTGGCTCAAACAAGAC TGGTTG-3′ (SEQ ID NO:1), flanked by SpeI and XhoI restriction enzyme recognition sequences. The isolated Grm6 enhancer element was fused to a SV40 eukaryotic promoter sequence from the pGL3-Promoter vector (Promega Corp.). The Grm6 enhancer-SV40 promoter fusion product was inserted into a modified (Matsuda and Cepko (2004) Proc. Natl. Acad. Sci. USA 101:16) pCAGGS vector (Niwa et al. (1991) Gene 108:193) lacking the CAG promoter. The cDNA encoding a ChR2-EYFP fusion protein was excised from the pLECYT lentiviral vector (Boyden et al. (2005) Nat. Neurosci. 8:1263) and cloned downstream of the Grm6 enhancer-SV40 minimal promoter elements to produce the pGrm6-CY expression plasmid. Subretinal injection and in vivo electroporation of the pGrm6-CY plasmid into newborn (P0 or P1) mouse pups was performed as previously described (Matsuda and Cepko, supra). Plasmid DNA was transfected into right eyes only.

Immunohistochemistry

Electroporated retinas were dissected from enucleated right eyes of sacrificed mice and fixed in 4% paraformaldehyde in phosphate buffered saline (PBS) for 30 minutes at room temperature, followed by incubation in PBS at 4° C. for 1-5 days. For wholemount preparations, retinas were flattened by immobilization on a nitrocellulose filter paper during the fixation and washing steps. For preparation of retinal sections, fixed and washed retinas were embedded in 2% agarose in PBS, and 200-μm vertical sections were cut with a Leica VT1000S vibratome. For immunohistochemistry, retinal wholemounts or sections were incubated with blocking solution (10% normal goat or donkey serum, 1% bovine serum albumin, 0.5% Triton X-100 in PBS [pH 7.4]) at room temperature for 1 hour. Retinal preparations were subsequently immunostained with polyclonal rabbit anti-GFP antibodies (1:200 dilution; Molecular Probes Inc.) or sheep anti-GFP antibodies (1:200 dilution; Biogenesis Inc.) for visualization of the ChR2-EYFP-expressing cells. To visualize retinal ganglion cells injected with neurobiotin during voltage-clamp experiments, retinas were co-stained with Alexa Fluor-conjugated streptavidin (1:200 dilution; Molecular Probes Inc.). Wholemount preparations were co-stained with anti-choline acetyl transferase antibodies (1:100 dilution; Chemicon International Inc.). Vertical sections were co-stained with mouse anti-PKC antibodies (1:100 dilution, BD Biosciences). After 3-7 days incubation, tissue preparations were washed three times in PBS at room temperature for at least 10 minutes per wash. Alexa Fluor-conjugated secondary antibodies (Molecular Probes Inc.) were applied at a dilution of 1:200 for 2 hours at room temperature, followed by three washes in PBS. Cell nuclei were stained with 10 μg/ml 4′,6-diamidino-2-phenylindole (DAPI) (Roche) overnight at room temperature. Following final washing in PBS, tissue preparations were mounted on slides with ProLong Gold antifade reagent (Molecular Probes Inc.).

Confocal Microscopy

Fluorescent specimens were viewed and confocal micrographs were taken using a Zeiss LSM 510 Meta Axioplan 2 laser scanning confocal microscope (Carl Zeiss Inc.) equipped with argon and helium-neon lasers and Plan-Apochromat 63×/1.4 or 100×/1.4 oil immersion objective lenses. Image reconstruction was done in Imaris (Bitplane Inc.). Quantification of bipolar cell stratification was performed using Matlab (Mathworks Inc.) and Mathematica 5.2 (Wolfram Research Inc.) software. Briefly, IPL boundaries were determined based on the positions of the INL and GCL visualized by nuclear staining with DAPI. The positions of labeled bipolar cell axon terminals within the IPL were calculated as a percentage of the distance between the IPL boundaries, with 0% representing the proximal IPL boundary (at the INL) and 100% representing the distal IPL boundary (at the GCL).

Multi-Electrode Array Recordings

Multi-electrode array recordings taken from whole-mount preparations of electroporated retinas revealed ChR2-mediated light responses in ON retinal ganglion cells. Excitatory signaling originating from ON bipolar cells was confirmed using pharmacological agents. Loose cell attached and whole cell voltage-clamp experiments demonstrated that ChR2-mediated light responses were generated during light ON in both ON (excitatory inputs) and OFF (inhibitory inputs) ganglion cells of pGrm6-CY electroporated cells.

To record the spike trains of retinal ganglion cells, the isolated retina of a wild-type C57BL/6J mouse, or mutant rd1 mouse electroporated with the pGrm6-CY construct was placed on a flat MEA60 200 Pt GND array (Ayanda Biosystems, Lausanne, Switzerland). The 30-μm diameter microelectrodes were spaced 200 μm apart on the array. The retina was continuously superfused in oxygenated Ringer's solution (110 mM NaCl, 2.5 mM KCl, 1.0 mM CaCl₂, 1.6 mM MgCl₂, 22 mM NaHCO₃, 10 mM D-glucose (pH 7.4 with 95% O₂ and 5% CO₂)) at 36° C. during experiments. Recordings ranged from 1-5 hours in duration, during which time ganglion cells maintained a stable average firing rate. The signals were recorded (MEA1060-2-BC, Multi-Channel Systems, Germany) and filtered between 500 Hz (low cut-off) and 3500 Hz (high cut-off). The spikes were extracted with a threshold of four times the standard deviation of the recorded trace and sorted with K-means algorithm based on the first three PCA components of 1-ms shape of the spike (Matlab; Mathworks Inc.).

### EXAMPLE 10

### Discussion

Several lines of evidence indicate that a core set of paired-type and POU homeodomain-containing transcription factors directly activate transcription of bipolar cell-expressed genes. Regulatory sequences of <500 bp were identified upstream of the mouse Grm6, Cabp5, and Chx10 genes that were capable of driving bipolar cell-specific reporter expression. These sequences each contained predicted paired-type and POU homeodomain-containing transcription factor binding sequences, and these sites were shown to be required, individually or in combination, for reporter expression. Sites upstream of each gene were also demonstrated to be able to interact with the POU homeodomain-containing transcription factor, Brn2, and the paired-type homeodomain-containing transcription factors, Crx and Otx2. Conditional inactivation of Otx2 led to loss of reporter expression, and dominant-negative Brn2-DBD-EnR expression reduced activity of two reporters. Endogenous Grm6, Cabp5, and Chx10 expression was reduced in Otx₂^{±} mutant retinas, and the expression of or the number of cells transcribing these genes was further reduced in Otx2^{-/-}; Crx^{-/-} retinas. Similar results were obtained for other bipolar cell-enriched genes, indicating the general importance of these paired-type homeodomain-containing transcription factors in bipolar cell gene expression.

It is possible that other transcription factors interact with the identified CREs. Evaluation of the importance of the predicted paired-type and POU homeodomain-containing transcription factor binding sequences was based primarily on recognition of these putative sites by a limited database of consensus sites of varying information content. However, even using this limited set, occurrence of binding sites for these transcription factor families was selective in that, for example, no putative LIM homeobox transcription factor binding sequences were found, despite representation in the database. Moreover, represented binding sites for other paired-type homeodomain-containing transcription factors, such as Chx10, were not observed. Even the sites identified using the limited database might also interact with related transcription factors other than Brn2, Crx, and Otx2, such as the bipolar cell-expressed transcription factors Chx10, Vsx1, and Og9x (Liu et al., 1994; Burmeister et al, 1996; Chow et al., 2001; Kim et al., 2007). More exhaustive EMSA analysis could aid in identifying other interacting transcription factors. However, none of these related transcription factors except Chx10 is expressed in as many bipolar cells as Otx2 (Koike et al., 2007), and thus activation of at least the pan-bipolar cell gene Chx10 would have to depend on additional proteins. It is also possible that CREs other than those isolated in this study regulate Grm6, Cabp5, and Chx10 expression. Indeed, a proximal Chx10 CRE has been characterized containing sequences directing dividing progenitor and bipolar cell expression that can be bound by Brn2 (Rowan and Cepko, 2005). The novel isolated Chx10 and Grm6 regulatory elements are positioned 19 and 8 kb upstream of transcriptional start sites, respectively. Other regulatory elements (e.g., upstream and/or downstream of the transcriptional start sites) in the vicinity of Grm6, Cabp5, and Chx10 could exist and contain sites for transcription factors not discussed here.

Consistent with a role for Otx2 in transcription of bipolar cell genes, Grm6, Cabp5, and Chx10 reporter activity and endogenous expression levels in the retina, as assessed by RNA in situ hybridization, were attenuated in Otx2^{±} and Otx2 CKO retinas. In contrast, a previous analysis found that Otx2^{±} mutants exhibit normal Chx10 retinal expression, as assessed by antibody staining (Koike et al., 2007). Differences in RNA in situ hybridization versus immunohistochemical results could reflect uncharacterized translational control and/or protein stability of Chx10. Further, it is possible that reduction of Grm6, Cabp5, and Chx10 reporter activity and endogenous expression levels reflects decrease of bipolar cell numbers in Otx2^{±} and Otx2 CKO retinas through effects on bipolar cell genesis or survival. However, previous analysis showed that Otx2^{±} retinas did not exhibit elevated cell death during retinogenesis (Koike et al., 2007). Additionally, Otx2 CKO retinas that were transfected with Cre exhibited virtually no Grm6, Cabp5, or Chx10 reporter activity, but co-transfected GFP-positive bipolar cells identified based on position and cell body morphology were still evident. Thus, without intending to be bound by scientific theory, while some bipolar cell death might have resulted from reduction of Otx2 function, Grm6, Cabp5, and Chx10 gene expression depends partially on Otx2, and this transcription factor likely directly activates transcription of these genes. Otx2^{±}; Crx^{-/-} mutant retinas were shown to exhibit elevated cell death, and so it is unclear whether further attenuation of hybridization signals for bipolar cell markers reflects a role for Crx in regulating bipolar cell genes or promoting survival in the Otx2^{±} mutant background (Koike et al., 2007).

The results also indicate that multiple regulatory elements for Chx10 exist for control of gene expression timing and/or cell-type specificity. The distal 164-bp Chx10 CRE is capable of driving reporter transcription in bipolar cells relatively late during retinogenesis and is incapable of activating detectable reporter expression in dividing progenitor cells. In contrast, the previously characterized, proximal Chx10 CRE is sufficient for reporter expression in bipolar cells and dividing progenitor cells in transgenic mice (Rowan and Cepko, 2005). Without intending to be bound by scientific theory, the role of the distal 164-bp Chx10 CRE might thus be to increase Chx10 transcription at a time when bipolar cells first appear and/or thereafter. In this regard, it is interesting that both regulatory elements contain Bm2 binding sites critical for reporter expression. Late activity of the distal 164-bp Chx10 CRE might result from the persistence of high levels of Otx2 only in developing bipolar cells in the retina as has been demonstrated previously (Baas et al., 2000; Koike et al., 2007). It was not possible to address whether Otx2 regulates the proximal CRE using electroporation. Surprisingly, this promoter was not active in transfected retinas. The proximal CRE was active in transgenic mice, which points to some differences due to integration or other aspects of DNA regulation associated with transgenesis (Rowan and Cepko, 2005).

Because Chx10 is necessary and sufficient for bipolar cell fate determination (Burmeister et al., 1996; Green et al., 2003; Livne-Bar et al., 2006), one possibility is that Otx2 might drive bipolar cell fate determination by activating the distal 164-bp Chx10 CRE and increasing Chx10 expression beyond the levels found in progenitor cells. This notion, coupled with results consistent with a role for Otx2 in directly activating transcription of differentiation genes, such as Grm6 and Cabp5, as well as Prkca (Koike et al., 2007), raises the possibility that Otx2 regulates both early bipolar cell fate determination genes and late differentiation genes in a relatively simple transcriptional hierarchy. Otx2 transcriptional regulation has been explored previously (Kurokawa et al., 2004), but factors driving relatively specific bipolar cell expression of Otx2 remain unidentified. An alternative possibility to direct regulation of early and late bipolar cell genes by Otx2 is that it could activate Chx10 expression, and Chx10 could then activate transcription of late differentiation genes, like Grm6 and Cabp5. Chx10 has been shown to function as a transcriptional repressor, and so putative Chx10 activation of bipolar cell genes would be expected to be indirect or reflect a dual ability to repress and activate in different contexts (Livne-Bar et al., 2006; Dorval et al., 2006; Dorval et al., 2005). It is also possible that Otx2 regulation of Chx10 gene expression does not play a role in bipolar cell fate determination.

The results also indicate that paired-type and POU homeodomain-containing transcription factors could act together to drive bipolar cell gene expression. Isolated regulatory elements were sensitive to deletions of binding sites for both paired-type and POU homeodomain-containing transcription factors. This indicates that action by transcription factors from these families is critical in driving transcription at least for these elements. Joint action of paired-type and POU homeodomain-containing transcription factors might aid in refining spatial and/or temporal expression. It might also provide quantitative control. For instance, it is apparent that while in Otx2 CKO and CAG-Bm2-DBD-EnR-transfected retinas the 164-bp Chx10 reporter expression decreased, some endogenous Chx10 expression persisted above the threshold necessary for bipolar cell development because transfected bipolar cells, identified based on position and cell body morphology, were still present. Although Otx2 and Brn2 are important for Chx10, Grm6, and Cabp5 expression, each is not sufficient as many cells contain these transcription factors but do not express these genes. For example, Otx2 is expressed throughout the developing forebrain, in the optic vesicle, in developing photoreceptor cells, and in the retinal pigmented epithelium (Acampora et al., 1995; Koike et al., 2007). Brn2 is found in the developing cortex and many early retinal progenitor cells (Sugitani et al., 2002; Rowan and Cepko, 2005). Additionally, in the developing neonatal rat retina, Otx2 misexpression led to greater photoreceptor cell production (Nishida et al., 2003). Other investigators found that XOtx2 misexpression in the developing Xenopus retina can promote the bipolar cell fate (Viczian et al., 2003).

The results have shed light on the core transcription factors that are important in driving expression of several bipolar cell genes. Determining transcription factors directing expression of Grm6, Cabp5, and Chx10 in different bipolar cell subtypes will be the subject of future studies. Differentially expressed transcription factors such as Vsx1, Isl1, Irx5, Bhlhb4, and Bhlhb5 could work in concert with Otx2 and/or Brn2 to shape bipolar cell subtype gene expression. Additionally, genomic sequences from other genes sufficient for specific bipolar cell expression have previously been identified (Oberdick et al., 1990; Wong et al., 1999). Unbiased screening methods for CREs described here will be used to isolate elements from these and other bipolar cell genes. Screening using a functional criterion was advantageous as compared to relying on a conserved sequence-based approach because many conserved sequences turned out to be dispensable for bipolar cell expression. Electroporation of constructs containing relatively short CREs inserted upstream of fluorescent reporters, transsynaptic tracing molecules, toxins, and/or activity-altering ion channels will prove useful for more precise mapping of retinal circuitry, study of retinal physiology, and cell-type-specific gene therapy using viral vectors.

### EXAMPLE 11

### Materials and Methods For Examples 1-8

Plasmid DNA Constructs

CAG-GFP and UB-GFP plasmids, in which contain broadly-active promoters that drive GFP expression, were from Matsuda and Cepko (2004). UB-tdTomato was constructed by excising the tdTomato cDNA from RSET-B-tdTomato (Shaner et al., 2004) and using it to replace the GFP sequence in UB-GFP. The 10-kb mouse genomic fragment (−9727 to +409 relative to the first nucleotide of BC021919, GenBank, NIH) Grm6-LacZ construct was the plasmid, MG6-Z, from Ueda et al. (1997). A BglII deletion construct (sequence left in construct: −9727 to −8127 and −2331 to +409) was made by BglII restriction enzyme digestion and re-ligation. An MscI deletion construct (sequence left in construct: −9727 to −7113 and −76 to +409) was made by Msc1 restriction enzyme digestion and re-ligation. The construct containing the 1-kb critical region and the 3′ 0.5-kb sequence (−8126 to −7113 and −76 to +409) was made by PCR amplification of genomic sequence from the MscI deletion construct using primers, 5-GATCTCCAGATGGCTAAAC-3′ (SEQ ID NO: 12) and 5′-GGCGGACGAAGCTGCCACCC-3′ (SEQ ID NO:13), and insertion of this fragment into the LacZ reporter vector. The construct containing the 1-kb critical region without the conserved 5′ sequence and with the 3′ 0.5-kb sequence (−7946 to −7113 and −76 to +409) was made by PCR amplification of genomic sequence from the MscI deletion construct using primers, 5-GGCTCAAACAAGACTGGTTG-3′ (SEQ ID NO: 14) and 5′-GGCGGACGAAGCTGCCACCC-3′ (SEQ ID NO:15), and insertion of this fragment into the LacZ reporter vector. The construct containing the 0.5-kb 3′ sequence alone (−76 to +409) was made by PCR amplification of genomic sequence from the original 10-kb construct using primers, 5-CCAAGCTTATTGGTGTTGC-3′ (SEQ ID NO:16) and 5′-GGCGGACGAAGCTGCCACCC-3′ (SEQ ID NO:17), and insertion of this fragment into the LacZ reporter vector. The SV40 basal promoter-LacZ construct was made by excising the SV40 basal promoter from the GL3 plasmid (Promega, Madison, Wis.) and inserting it into the LacZ reporter vector. The construct containing the 200-bp region (−8126 to −7927) and the SV40 basal promoter was made by PCR amplification of a fragment from mouse genomic DNA using primers, 5′-GATCTCCAGATGGCTAAAC-3′ (SEQ ID NO:18) and 5′-CAACCAGTCTTGTTTGAGCC-3′ (SEQ ID NO:19), and insertion of this fragment into the SV40 basal promoter-LacZ vector.

The 4.7-kb mouse genomic fragment (−4529 to +156 relative to the first nucleotide of NM_{—}013877) from the Cabp5 gene was inserted upstream of GFP or tdTomato by excision from the Cabp5-dsRed plasmid (Matsuda and Cepko, 2004) and insertion into the UB-GFP or UB-tdTomato constructs, replacing the human ubiquitin C promoter. The construct containing the 445-bp sequence (−289 to +156) and GFP or tdTomato was made by PCR amplification of a fragment from mouse genomic DNA using primers, 5′-GCATCTTGTTCCTTTGGGCG-3′ (SEQ ID NO:20) and 5′-CATTGGAGCAGGTAGTG-3′ (SEQ ID NO:21), and insertion of this fragment into the UB-GFP or UB-tdTomato constructs, replacing the human ubiquitin C promoter.

The 210-kb Chx10-containing plasmid (−109,003 to +101,164 relative to the first nucleotide of NM_{—}007701) was bacterial artificial chromosome (BAC) RP23-240D15 (BACPAC Resources, Children's Hospital Oakland Research Institute, Oakland, Calif.). For unbiased CRE screening, an SV40 basal promoter-GFP construct was made by excising the SV40 basal promoter from the GL3 plasmid and inserting it into the UB-GFP construct, replacing the human ubiquitin C promoter. For CRE library construction, approximately 200 ng of EcoRI-digested BAC DNA was ligated with approximately 10 ng of digested SV40 basal promoter-GFP vector. An alkaline phosphatase (AP) reporter construct was made by inserting EMCV IRES (Matusda and Cepko, 2004) and human placental AP (Fields-Berry et al., 1992) sequences into the SV40 basal promoter-GFP vector downstream of the GFP sequence. Similar to this SV40 basal promoter-GFP-IRES-AP vector, an SV40 basal promoter-tdTomato-IRES-AP construct was also made. The constructs containing the 164-bp region (−17,748 to −17,585) and the SV40 basal promoter were made by PCR amplification of a fragment from mouse genomic DNA using primers, 5′-GAGAAGAGCACTGGCTGGGG-3′ (SEQ ID NO:22) and 5′-AATTCCATTTGATGCATTAGAACTAATTCTCCTCC-3′ (SEQ ID NO:23), and insertion of this fragment into the SV40 basal promoter-GFP-IRES-AP or SV40 basal promoter-tdTomato-IRES-AP vector. Grm6, Cabp5, and Chx10 CRE deletion constructs were made using PCR-based mutagenesis to remove sequences detailed above.

A CAG-Brn2 construct was made by PCR amplification of a fragment from mouse retinal cDNA using primers, 5′-CATGGCGACCGCAGCGTCTAACC-3′ (SEQ ID NO:24) and 5′-TCACTGGACGGGCGTCTGCAC-3′ (SEQ ID NO:25), and insertion of this fragment into the CAG-GFP vector, replacing the GFP sequence. A CAG-CrxMyc construct was made by PCR amplification of a fragment from mouse retinal cDNA using primers, 5′-GTGTGAGGGGACCTATTTCC-3′ (SEQ ID NO:26) and 5′-CAAGATCTGAAACTTCCAGG-3′ (SEQ ID NO:27), and insertion of this fragment and a C-terminal Myc tag sequence (gaacaaaaacttatttctgaagaagatctgtg) (SEQ ID NO:28) into the CAG-GFP vector, replacing the GFP sequence. A CAG-Otx2Myc construct was made by PCR amplification of a fragment from mouse retinal cDNA using primers, 5′-CTGGAACGTGGAGGAAGCTG-3′ (SEQ ID NO:29) and 5′-CAAAACCTGGAATTTCCATG-3′ (SEQ ID NO:30), and insertion of this fragment and a C-terminal Myc tag sequence into the CAG-GFP vector, replacing the GFP sequence. CAG-Cre was from Matsuda and Cepko (2007). The dominant-negative CAG-Brn2-DBD-EnR construct was made by PCR amplification of a fragment from mouse retinal cDNA using primers, 5′-CCATGGGCACGCCGACCTCAGACGACCTGGAGC-3′ (SEQ ID NO:31) and 5′-ACCGGTCCGGGAGGGGTCATCCTTTTCTC-3′ (SEQ ID NO:32), and insertion of this fragment and a C-terminal engrailed repressor domain (EnR; Conlon et al., 1996) into the CAG-GFP vector, replacing the GFP sequence.

DNA Transfection of Retinas by Electroporation

DNA transfection by in vivo electroporation was carried out as in Matsuda and Cepko (2004). For co-transfection, equimolar quantities of plasmid were used, and DNA concentration per plasmid was approximately 2-4 mg/mL. The injection volume was 0.2 μL. DNA transfection by in vitro electroporation was performed as in Kim et al. (2007). For unbiased CRE screening, miniprep DNA was used (approximately 0.1 mg/mL). For all other in vitro electroporations, DNA concentration per plasmid was approximately 1-2 mg/mL. The volume for in vitro electroporations was 70 μL.

Preparation of Retinal Sections

For experiments where histochemical staining was not performed, harvested retinas were dissected or removed from culture and rinsed in PBS (pH 7.4), fixed in 4% paraformaldehyde in PBS for 30 min at 22° C., rinsed three times, and cryoprotected for 1 h in 30% sucrose in PBS. Retinas were embedded in OCT (Sakura Finetek, Torrance, Calif.), and 20-μm sections were cut and slide-mounted using a cryostat microtome.

Bioinformatic Sequence Analysis

Sequence analysis was based on the February 2006 (mm8) mouse genome assembly from the UCSC Genome Browser Project (Santa Cruz, Calif.; Kent et al., 2002). Output of the phastCons program downloaded from the UCSC Genome Browser was used initially to compare syntenic sequences across genomes of different species (Siepel et al., 2005; Karolchik et al., 2003). The rVista program (version 2.0; Loots and Ovcharenko, 2004) was used to filter isolated CRE sequences through the TRANSFAC database (version 10.2; Matys et al., 2006) of 467 vertebrate transcription factor binding sequences. Individual mouse sequences were submitted to rVista using the zPicture program, and thresholds for sequence match to TRANSFAC entries were set so that they were optimized for function (Ovcharenko et al., 2004). Percent identity of mouse CRE sequences to those in other genomes was calculated after alignment using CLUSTALW (Larkin et al, 2007).

Electrophoretic Mobility Shift Assays

Approximately 1×10⁶ 293T cells transfected for 36 h with 3 μg CAG-GFP, CAG-Brn2, CAG-CrxMyc, or CAG-Otx2Myc plasmid DNA using Lipofectamine 2000 (Invitrogen, Carlsbad, Calif.) according to the manufacturer's protocol were utilized. Nuclear extracts were prepared from these cells or adult mouse retinas using NE-PER nuclear and cytoplasmic extraction reagents (Pierce, Rockford, Ill.) according to the manufacturer's protocol. Complementary oligonucleotides were annealed to make double-stranded probes with tetranucleotide overhangs. These were end-labeled with [α-³²P]dCTP (GE Healthcare, Piscataway, N.J.) using Klenow enzyme (Roche, Indianapolis, Ind.). Probes were purified of free nucleotide using Sephadex G-25 spin columns (Roche). Binding reactions were performed for 30 min at 22° C. using 1 μg of nuclear extract protein and approximately 2×10⁵ CPM of probe in 10% glycerol, 10 mM Tris (pH 7.5), 50 mM KCl, 0.5 mM DTT, and 3 μg of poly(dI-dC). Binding reactions were electrophoresed on 6% polyacrylamide gels (Invitrogen) buffered in 0.5× TBE at 200 V for 30 min. Dried gels were exposed to Amersham Hyperfilm MP (GE Healthcare) for 1 hour for transfected cell nuclear extracts or 16 hours for retinal nuclear extracts. These and complementary oligonucleotides with overhangs were used: Grm6 Pax6 site—5′-ctagCTAAACTTTTAAATCATGAATGAAGTAGA-3′ (SEQ ID NO:33); Grm6 Pou3f2 site—5′-ctagCTTTAATCTGTTAATGTAGT-3′ (SEQ ID NO:34); Grm6 Crx site—5′-ctagCAAATCGTTAATCTGCTAAAG-3′ (SEQ ID NO:35); Cabp5 Crx site—5′-ctagCCTCACCCTAATCCCTCTTTC-3′ (SEQ ID NO:36); Cabp5 5′ Brn2 site—5′-ctagCCCTCTTTCAAAATGTACTATC-3′ (SEQ ID NO:37); Cabp5 Pitx2 site—5′-ctagAGAGCTCTAATCCCTCCACT-3′ (SEQ ID NO:38); Cabp5 3′ Brn2 site—5′-ctagTAAGTAGAATTTTCCATGAGCTGT-3′ (SEQ ID NO:39); Chx10 Crx site—5′-ctagTTGCCCGCTAATCCCAGCTG-3′ (SEQ ID NO:40); Chx10 Pou3f2 site—5′-ctagCTGCCATTAAAATATTAAAG-3′ (SEQ ID NO:41); Chx10 Otx site—5′-ctagAGATAAATCTAATCGTCTCT-3′ (SEQ ID NO:42); and Chx10 Brn2 site—5′-ctagTCTCTTTATCCAAAATAAGCGACT-3′ (SEQ ID NO:43).

Western Blots

Nuclear extract protein (1 μg) was subjected to SDS-PAGE using 4-20% Tris-glycine gels (Invitrogen) and transferred to nitrocellulose filters (Invitrogen). Membranes were probed with a goat polyclonal anti-Brn2 antibody (1:500, sc-6029, Santa Cruz Biotechnology, Santa Cruz, Calif.) or a mouse monoclonal anti-Myc antibody (1:500, 9E10, sc-40, Santa Cruz Biotechnology) and then with horseradish peroxidase-conjugated donkey anti-goat (1:5000, Jackson ImmunoResearch Laboratories, West Grove, Pa.) or goat anti-mouse (1:5000, Jackson ImmunoResearch Laboratories) antibodies. Immunoreactivity was revealed using enhanced chemiluminescence detection reagents (GE Healthcare).

Histochemical Staining

To assess β-galactosidase activity, 5-bromo-4-chloro-3-indolyl-β-D-galactopyranoside (X-gal; Research Organics, Cleveland, Ohio) staining was carried out as in Furukawa et al. (2002), except retinas were fixed in 4% paraformaldehyde in PBS for 30 min at 22° C. Retinas were stained for 16 hours at 37° C. To assess AP activity, staining with 5-bromo-4-chloro-3-indolyl-phosphate (BCIP; Sigma, St. Louis, Mo.) and nitroblue tetrazolium (NBT, Sigma) was performed as in Fields-Berry et al. (1992), except retinas were fixed in 4% paraformaldehyde in PBS for 5 minutes on ice. Retinas were stained for 16 hours at 37° C. Stained retinas were then sectioned as above.

RNA In Situ Hybridization

Hybridization of riboprobes to retinal sections was carried out as in Murtaugh et al. (1999) with modifications detailed in Trimarchi et al. (2007). Riboprobes used are listed in Kim et al. (2007).

Animals

Wild-type neonates used for electroporation were obtained from pregnant Sprague-Dawley rats (Taconic Farms, Hudson, N.Y.) and CD-1 mice (Charles River Laboratories, Wilmington, Mass.). Otx2^{flox/flox} mice were obtained from S. Aizawa (Tian et al., 2002; RIKEN Center for Developmental Biology, Kobe, Japan). An Otx2 null allele resulted from mating to human β-actin:Cre transgenic deleter mice (Lewandoski et al., 2007; The Jackson Laboratory, Bar Harbor, Me.), and mice carrying this mutation were then crossed with Crx^{-/-} mice (Furukawa et al., 1999). Intercrosses of Otx2^{±}; Crx^{±} mice led to generation of wild-type, Otx2^{±}, Crx^{-/-}, and Otx2^{±}; Crx^{-/-} mice used for RNA in situ hybridization. All animals were used in accordance with the guidelines for animal care and experimentation established by the National Institutes of Health and the Harvard Medical Area Standing Committee on Animals.

### EXAMPLE 12

### REFERENCES

Acampora D, Mazan S, Lallemand Y, Avantaggiato V, Maury M, Simeone A, Brûlet P (1995) “Forebrain and midbrain regions are deleted in Otx2−/− mutants due to a defective anterior neuroectoderm specification during gastrulation,” Development 121:3279-3290.

Akagi T, Inoue T, Miyoshi G, Bessho Y, Takahashi M, Lee J E, Guillemot F, Kageyama R (2004) “Requirement of multiple basic helix-loop-helix genes for retinal neuronal subtype specification,” J. Biol. Chem. 279:28492-28498.

Baas D, Bumsted K M, Martinez J A, Vaccarino F M, Wikler K C, Barnstable C J (2000) “The subcellular localization of Otx2 is cell-type specific and developmentally regulated in the mouse retina,” Brain Res. Mol. Brain Res. 78:26-37.

Berrebi A S, Oberdick J, Sangameswaran L, Christakos S, Morgan J I, Mugnaini E (1991) “Cerebellar Purkinje cell markers are expressed in retinal bipolar neurons,” J. Comp. Neurol. 308:630-649.

Blackshaw S, Harpavat S, Trimarchi J, Cai L, Huang H, Kuo W P, Weber G, Lee K, Fraioli R E, Cho S H, Yung R, Asch E, Ohno-Machado L, Wong W H, Cepko C L (2004) “Genomic analysis of mouse retinal development,” PLoS. Biol. 2:E247.

Bramblett D E, Pennesi M E, Wu S M, Tsai M J (2004) “The transcription factor Bhlhb4 is required for rod bipolar cell maturation,” Neuron 43:779-793.

Burmeister M, Novak J, Liang M Y, Basu S, Ploder L, Hawes N L, Vidgen D, Hoover F, Goldman D, Kalnins V I, Roderick T H, Taylor B A, Hankin M H, McInnes R R (1996) “Ocular retardation mouse caused by Chx10 homeobox null allele: impaired retinal progenitor proliferation and bipolar cell differentiation,” Nat. Genet. 12:376-384.

Chen S, Wang Q L, Nie Z, Sun H, Lennon G, Copeland N G, Gilbert D J, Jenkins N A, Zack D J (1997) “Crx, a novel Otx-like paired-homeodomain protein, binds to and transactivates photoreceptor cell-specific genes,” Neuron 19:1017-1030.

Cheng C W, Chow R L, Lebel M, Sakuma R, Cheung H O, Thanabalasingham V, Zhang X, Bruneau B G, Birch D G, Hui C C, McInnes R R, Cheng S H (2005) “The Iroquois homeobox gene, Irx5, is required for retinal cone bipolar cell development,” Dev. Biol. 287:48-60.

Chow R L, Snow B, Novak J, Looser J, Freund C, Vidgen D, Ploder L, McInnes RR (2001) “Vsx1, a rapidly evolving paired-like homeobox gene expressed in cone bipolar cells,” Mech. Dev. 109:315-322.

Chow R L, Volgyi B, Szilard R K, Ng D, McKerlie C, Bloomfield S A, Birch D G, McInnes R R (2004) “Control of late off-center cone bipolar cell differentiation and visual signaling by the homeobox gene Vsx1,” Proc. National. Acad. Sci. USA 101:1754-1759.

Clark A M, Yun S, Veien E S, Wu Y Y, Chow R L, Dorsky R I, Levine E M (2007) “Negative regulation of Vsx1 by its paralog Chx10Vsx2 is conserved in the vertebrate retina,” Brain Res., in press.

Conlon F L, Sedgwick S G, Weston K M, Smith J C (1996) “Inhibition of Xbra transcription activation causes defects in mesodermal patterning and reveals autoregulation of Xbra in dorsal mesoderm,” Development 122:2427-2435.

Dorval K M, Bobechko B P, Ahmad K F, Bremner R (2005) “Transcriptional activity of the paired-like homeodomain proteins CHX10 and VSX1,” J. Biol. Chem. 280:10100-10108.

Dorval K M, Bobechko B P, Fujieda H, Chen S, Zack D J, Bremner R (2006) “CHX10 targets a subset of photoreceptor genes,” J. Biol. Chem. 281:744-751.

Elshatory Y, Everhart D, Deng M, Xie X, Barlow R B, Gan L (2007) “Islet-1 controls the differentiation of retinal bipolar and cholinergic amacrine cells,” J. Neurosci. 27:12707-12720.

Euler T, Wässle H (1995) “Immunocytochemical identification of cone bipolar cells in the rat retina,” J. Comp. Neurol. 361:461-478.

Feng L, Xie X, Joshi P S, Yang Z, Shibasaki K, Chow R L, Gan L (2006) “Requirement for Bhlhb5 in the specification of amacrine and cone bipolar subtypes in mouse retina,” Development 133:4815-4825.

Fields-Berry S C, Halliday A L, Cepko C L (1992) “A recombinant retrovirus encoding alkaline phosphatase confirms clonal boundary assignment in lineage analysis of murine retina,” Proc. Natl. Acad. Sci. USA 89:693-697.

Fletcher E L, Koulen P, Wassle H (1998) “GABAA and GABAC receptors on mammalian rod bipolar cells,” J. Comp. Neurol. 396:351-365.

Furukawa A, Koike C, Lippincott P, Cepko C L, Furukawa T (2002) “The mouse Crx 5′-upstream transgene sequence directs cell-specific and developmentally regulated expression in retinal photoreceptor cells,” J. Neurosci. 22:1640-1647.

Furukawa T, Morrow E M, Cepko C L (1997) “Crx, a novel otx-like homeobox gene, shows photoreceptor-specific expression and regulates photoreceptor differentiation,” Cell 91:531-541.

Furukawa T, Morrow E M, Li T, Davis F C, Cepko C L (1999) “Retinopathy and attenuated circadian entrainment in Crx-deficient mice,” Nat. Genet. 23:466-470.

Ghosh K K, Bujan S, Haverkamp S, Feigenspan A, Wassle H (2004) “Types of bipolar cells in the mouse retina,” J. Comp. Neurol. 469:70-82.

Gray P A, Fu H, Luo P, Zhao Q, Yu J, Ferrari A, Tenzen T, Yuk D I, Tsung E F, Cai Z, Alberta J A, Cheng L P, Liu Y, Stenman J M, Valerius M T, Billings N, Kim H A, Greenberg M E, McMahon A P, Rowitch D H, Stiles C D, Ma Q (2004) “Mouse brain organization revealed through direct genome-scale transcription factor expression analysis,” Science 306:2255-2257.

Green E S, Stubbs J L, Levine E M (2003) “Genetic rescue of cell number in a mouse model of microphthalmia: interactions between Chx10 and G1-phase cell cycle regulators,” Development 130:539-552.

Greferath U, Grünert U, Wässle H (1990) “Rod bipolar cells in the mammalian retina show protein kinase C-like immunoreactivity,” J. Comp. Neurol. 301:433-442.

Haeseleer F, Sokal I, Verlinde C L, Erdjument-Bromage H, Tempst P, Pronin A N, Benovic J L, Fariss R N, Palczewski K (2000) “Five members of a novel Ca(2+)-binding protein (CABP) subfamily with similarity to calmodulin,” J. Biol. Chem. 275:1247-1260.

Haverkamp S, Ghosh K K, Hirano A A, Wässle H (2003a) “Immunocytochemical description of five bipolar cell types of the mouse retina,” J. Comp. Neurol. 455:463-476.

Haverkamp S, Haeseleer F, Hendrickson A (2003b) “A comparison of immunocytochemical markers to identify bipolar cell types in human and monkey retina,” Vis. Neurosci. 20:589-600.

He X, Treacy M N, Simmons D M, Ingraham H A, Swanson L W, Rosenfeld M G (1989) “Expression of a large family of POU-domain regulatory genes in mammalian brain development,” Nature 340:35-41.

Huang L, Max M, Margolskee R F, Su H, Masland R H, Euler T (2003) “G protein subunit G gamma 13 is coexpressed with G alpha o, G beta 3, and G beta 4 in retinal ON bipolar cells,” J. Comp. Neurol. 455:1-10.

Karolchik D, Baertsch R, Diekhans M, Furey T S, Hinrichs A, Lu Y T, Roskin K M, Schwartz M, Sugnet C W, Thomas D J, Weber R J, Haussler D, Kent W J; University of California Santa Cruz (2003) “The UCSC Genome Browser Database,” Nucleic Acids Res. 31:51-54.

Kent W J, Sugnet C W, Furey T S, Roskin K M, Pringle T H, Zahler A M, Haussler D (2002) “The human genome browser at UCSC,” Genome Res. 12:996-1006.

Kim D S, Ross S E, Trimarchi J M, Aach J, Greenberg M E, Cepko C L (2007) “Identification of molecular markers of bipolar cells in the murine retina,” J. Comp. Neurol., in press.

Koike C, Nishida A, Ueno S, Saito H, Sanuki R, Sato S, Furukawa A, Aizawa S, Matsuo I, Suzuki N, Kondo M, Furukawa T (2007) “Functional roles of Otx2 transcription factor in postnatal mouse retinal development,” Mol. Cell Biol. 27:8318-8329.

Koulen P, Brandstätter J H, Enz R, Bormann J, Wässle H (1998) “Synaptic clustering of GABA(C) receptor rho-subunits in the rat retina,” Eur. J. Neurosci. 10:115-127.

Kurokawa D, Kiyonari H, Nakayama R, Kimura-Yoshida C, Matsuo I, Aizawa S (2004) “Regulation of Otx2 expression and its functions in mouse forebrain and midbrain,” Development 131:3319-3331.

Larkin M A, Blackshields G, Brown N P, Chenna R, McGettigan P A, McWilliam H, Valentin F, Wallace I M, Wilm A, Lopez R, Thompson J D, Gibson T J, Higgins D G (2007) “Clustal W and Clustal X version 2.0,” Bioinformatics 23:2947-2948.

Laughon A (1991) “DNA binding specificity of homeodomains,” Biochemistry 30:11357-11367.

Lewandoski M, Meyers E N, Martin G R (1997) “Analysis of Fgf8 gene function invertebrate development,” Cold Spring Harb. Symp. Quant. Biol. 62:159-68.

Liu I S, Chen J D, Ploder L, Vidgen D, van der Kooy D, Kalnins V I, McInnes R R (1994) “Developmental expression of a novel murine homeobox gene (Chx10): evidence for roles in determination of the neuroretina and inner nuclear layer,” Neuron 13:377-393.

Livne-Bar I, Pacal M, Cheung M C, Hankin M, Trogadis J, Chen D, Dorval K M, Bremner R (2006) “Chx 10 is required to block photoreceptor differentiation but is dispensable for progenitor proliferation in the postnatal retina,” Proc. Natl. Acad. Sci. USA 103:4988-4993

Loots G G, Ovcharenko I (2004) “rVISTA 2.0: evolutionary analysis of transcription factor binding sites,” Nucleic Acids Res. 32:W217-W221.

Matsuda T, Cepko C L (2004) “Electroporation and RNA interference in the rodent retina in vivo and in vitro,” Proc. Natl. Acad. Sci. USA 101:16-22.

Matsuda T, Cepko C L (2007) “Controlled expression of transgenes introduced by in vivo electroporation,” Proc. Natl. Acad. Sci. USA 104:1027-1032

Matys V, Kel-Margoulis O V, Fricke E, Liebich I, Land S, Barre-Dirrie A, Reuter I, Chekmenev D, Krull M, Homischer K, Voss N, Stegmaier P, Lewicki-Potapov B, Saxel H, Kel A E, Wingender E (2006) “TRANSFAC and its module TRANSCompel: transcriptional gene regulation in eukaryotes,” Nucleic Acids Res. 34:D108-D110.

Murtaugh L C, Chyung J H, Lassar A B (1999) “Sonic hedgehog promotes somitic chondrogenesis by altering the cellular response to BMP signaling,” Genes Dev. 13:225-237.

Nakajima Y, Iwakabe H, Akazawa C, Nawa H, Shigemoto R, Mizuno N, Nakanishi S (1993) “Molecular characterization of a novel retinal metabotropic glutamate receptor mGluR6 with a high agonist selectivity for L-2-amino-4-phosphonobutyrate,” J. Biol. Chem. 268:11868-11873.

Nishida A, Furukawa A, Koike C, Tano Y, Aizawa S, Matsuo I, Furukawa T (2003) “Otx2 homeobox gene controls retinal photoreceptor cell fate and pineal gland development,” Nat. Neurosci. 6:1255-1263.

Oberdick J, Smeyne R J, Mann J R, Zackson S, Morgan J I (1990) “A promoter that drives transgene expression in cerebellar Purkinje and retinal bipolar neurons,” Science 248:223-226.

Ohtoshi A, Justice M J, Behringer R R (2001) “Isolation and characterization of Vsx1, a novel mouse CVC paired-like homeobox gene expressed during embryogenesis and in the retina,” Biochem. Biophys. Res. Commun. 286:133-140.

Ovcharenko I, Loots G G, Hardison R C, Miller W, Stubbs L (2004) “zPicture: dynamic alignment and visualization tool for analyzing conservation profiles,” Genome Res. 14:472-477.

Pignatelli V, Strettoi E (2004) “Bipolar cells of the mouse retina: a gene gun, morphological study,” J. Comp. Neurol. 476:254-266.

Rowan S, Cepko C L (2004) “Genetic analysis of the homeodomain transcription factor Chx10 in the retina using a novel multifunctional BAC transgenic mouse reporter,” Dev. Biol. 271:388-402.

Rowan S, Cepko C L (2005) “A POU factor binding site upstream of the Chx10 homeobox gene is required for Chx10 expression in subsets of retinal progenitor cells and bipolar cells,” Dev. Biol. 281:240-255.

Sato S, Inoue T, Terada K, Matsuo I, Aizawa S, Tano Y, Fujikado T, Furukawa T (2007) “Dkk3-Cre BAC transgenic mouse line: a tool for highly efficient gene deletion in retinal progenitor cells,” Genesis 45:502-507.

Shaner N C, Campbell R E, Steinbach P A, Giepmans B N, Palmer A E, Tsien R Y (2004) “Improved monomeric red, orange and yellow fluorescent proteins derived from Discosoma sp. red fluorescent protein,” Nat. Biotechnol. 22:1567-72.

Siepel A, Bejerano G, Pedersen J S, Hinrichs A S, Hou M, Rosenbloom K, Clawson H, Spieth J, Hillier L W, Richards S, Weinstock G M, Wilson R K, Gibbs R A, Kent W J, Miller W, Haussler D (2005) “Evolutionarily conserved elements in vertebrate, insect, worm, and yeast genomes,” Genome Res. 15:1034-1050.

Sugitani Y, Nakai S, Minowa O, Nishi M, Jishage K, Kawano H, Mori K, Ogawa M, Noda T (2002) “Brn-1 and Brn-2 share crucial roles in the production and positioning of mouse neocortical neurons,” Genes Dev. 16:1760-1765.

Takebayashi K, Takahashi S, Yokota C, Tsuda H, Nakanishi S, Asashima M, Kageyama R (1997) “Conversion of ectoderm into a neural fate by ATH-3, a vertebrate basic helix-loop-helix gene homologous to Drosophila proneural gene atonal,” EMBO J. 16:384-395.

Tian E, Kimura C, Takeda N, Aizawa S, Matsuo I (2002) “Otx2 is required to respond to signals from anterior neural ridge for forebrain specification,” Dev. Biol. 242:204-223.

Tomita K, Moriyoshi K, Nakanishi S, Guillemot F, Kageyama R (2000) “Mammalian achaete-scute and atonal homologs regulate neuronal versus glial fate determination in the central nervous system,” EMBO J. 19:5460-5472.

Trimarchi J M, Stadler M B, Roska B, Billings N, Sun B, Bartch B, Cepko C L (2007) “Molecular heterogeneity of developing retinal ganglion and amacrine cells revealed through single cell gene expression profiling,” J. Comp. Neurol. 502:1047-1065.

Ueda Y, Iwakabe H, Masu M, Suzuki M, Nakanishi S (1997) “The mGluR6 5′ upstream transgene sequence directs a cell-specific and developmentally regulated expression in retinal rod and ON-type cone bipolar cells,” J. Neurosci. 17:3014-3023.

Vardi N (1998) “Alpha subunit of Go localizes in the dendritic tips of ON bipolar cells,” J. Comp. Neurol. 395:43-52.

Vardi N, Morigiwa K (1997) “ON cone bipolar cells in rat express the metabotropic receptor mGluR6,” Vis. Neurosci. 14:789-794.

Viczian A S, Vignali R, Zuber M E, Barsacchi G, Harris W A (2003) “XOtx5b and XOtx2 regulate photoreceptor and bipolar fates in the Xenopus retina,” Development 130:1281-1294.

Wong G T, Ruiz-Avila L, Margolskee R F (1999) “Directing gene expression to gustducin-positive taste receptor cells,” J. Neurosci. 19:5802-5809.

Young R W (1985) “Cell differentiation in the retina of the mouse,” Anat. Rec. 212:199-205.

What is claimed:

1. An isolated nucleic acid molecule comprising a nucleotide sequence which is at least 90% identical to the nucleotide sequence set forth as SEQ ID NO:1, 2 or 3.

2. The isolated nucleic acid molecule of claim 1, wherein the nucleotide sequence is at least 92% identical to the nucleotide sequence set forth as SEQ ID NO:1, 2 or 3.

3. The isolated nucleic acid molecule of claim 1, wherein the nucleotide sequence is at least 95% identical to the nucleotide sequence set forth as SEQ ID NO:1, 2 or 3.

4. The isolated nucleic acid molecule of claim 1, capable of directing bipolar cell-specific expression in a retina.

5. The isolated nucleic acid molecule of claim 4, further comprising a basal promoter sequence.

6. The isolated nucleic acid molecule of claim 1, having one or more POU homeodomain binding sites.

7. The isolated nucleic acid molecule of claim 1, having one or more paired-type homeodomain binding sites.

8. An isolated nucleic acid molecule comprising the nucleotide sequence set forth as SEQ ID NO:1, 2 or 3.

9. A host cell which contains the nucleic acid molecule of claim 4.

10. The isolated nucleic acid molecule of claim 4, operably linked to a nucleic acid sequence of interest, wherein the isolated nucleic acid molecule is capable of directing bipolar cell-specific expression of the nucleic acid sequence of interest.

11. The isolated nucleic acid molecule of claim 10, wherein the nucleic acid sequence of interest encodes an endogenous retinal mRNA sequence.

12. The isolated nucleic acid molecule of claim 10, wherein the nucleic acid sequence of interest includes a basal promoter sequence.

13. The isolated nucleic acid molecule of claim 10, wherein the nucleic acid sequence of interest encodes an antisense RNA sequence, an RNAi sequence or an siRNA sequence.

14. A gene therapy vector which comprises the nucleic acid molecule of claim 10.

15. A nucleic acid molecule comprising a fragment of at least 15 nucleotides of the nucleotide sequence set forth as SEQ ID NO:1, 2 or 3.

16. A method for treating a subject in need thereof comprising:
administering to the subject the isolated nucleic acid molecule of claim 4;
expressing the nucleic acid sequence of interest in a retinal cell; and
treating or preventing a retinal disorder.

17. The method of claim 16, wherein the retinal disorder is selected from the group consisting of atrophic macular degeneration, retinitis pigmentosa, iatrogenic retinopathy, retinal tears and holes, diabetic retinopathy, sickle cell retinopathy, retinal vein occlusion, retinal artery occlusion and aberrant retinal cell proliferation.

18. The method of claim 16, wherein the isolated nucleic acid molecule is administered using a gene therapy vector.

19. The method of claim 18, wherein the gene therapy vector includes a basal promoter sequence.

20. The method of claim 16, wherein the isolated nucleic acid molecule is administered by in vivo electroporation.
